SEQUENCE LISTING (93 sequences)

SEQ ID NO: 1 - DNA, 573 bp, Glycine max
  source 1..573: mol_type = genomic DNA
atgcgttatttgtctctgagtactttttctctcttgtgcattacctttgtggtcatggcagcctccgtggatcccctagtggttggtcgcgtgatcggcgatgtggtagacatgttcattccttcagtcaacatgtccgtttactttgggtcgaagcacgtcacaaatggctgtgacatcaagccatccattgccatcagccctcctaagctcaccctcaccggcaacatggataacctctacacactggttatgactgatcctgacgcacctagccccagtgaaccaagcatgcgcgagtggatacattggatcttagttgacatacctggaggaacaaacccatttcgcggaaaagagattgtttcatatgtgggaccaagaccacctattggaatacatcgctatatctttgtgttgtttcaacagaaaggacctttaggtcttgtggagcaaccaccaactcgagcaagcttcaacactcgttattttgccaggcaattggacttgggacttccagtggccactgtctacttcaactctcaaaaagaacctgctgttaagaggcgctga

SEQ ID NO: 2 - protein, 190 aa, Glycine max
MRYLSLSTFSLLCITFVVMAASVDPLVVGRVIGDVVDMFIPSVNMSVYFGSKHVTNGCDIKPSIAISPPKLTLTGNMDNLYTLVMTDPDAPSPSEPSMREWIHWILVDIPGGTNPFRGKEIVSYVGPRPPIGIHRYIFVLFQQKGPLGLVEQPPTRASFNTRYFARQLDLGLPVATVYFNSQKEPAVKRR

SEQ ID NO: 3 - DNA, 573 bp, Glycine max
  source 1..573: mol_type = genomic DNA
atgcgttatttgtctctgagtactttttctctcttgtgcattacctttgtggtcatggcagcctccgtggatcccctagtggttggtcgcgtgatcggcgatgtggtagacatgttcattccttcagtcaacatgtccgtttactttgggtcgaagcacgtcacaaatggctgtgacatcaagccatccattgccatcagccctcctaagctcaccctcaccggcaacatggataacctctacacactggttatgactgatcctgacgcacctagccccagtgaaccaagcatgcgcgagtggatacattggatcttagttgacatacctggaggaacaaacccatttcgcggaaaagagattgtttcatatgtgggaccaagaccacctattggaatacatcgctatatctttgtgtcctttcaacagaaaggacctttaggtcttgtggagcaaccaccaactcgagcaagcttcaacactcgttattttgccaggcaattggacttgggacttccagtggccactgtctacttcaactctcaaaaagaacctgctgttaagaggcgctga

SEQ ID NO: 4 - protein, 190 aa, Glycine max
MRYLSLSTFSLLCITFVVMAASVDPLVVGRVIGDVVDMFIPSVNMSVYFGSKHVTNGCDIKPSIAISPPKLTLTGNMDNLYTLVMTDPDAPSPSEPSMREWIHWILVDIPGGTNPFRGKEIVSYVGPRPPIGIHRYIFVSFQQKGPLGLVEQPPTRASFNTRYFARQLDLGLPVATVYFNSQKEPAVKRR

SEQ ID NO: 5 - DNA, 573 bp, Glycine max
  source 1..573: mol_type = genomic DNA
atgcgttatttgtctctgagtactttttctctcttgtgcattacctttgtggtcatggcagcctccgtggatcccctagtggttggtcgcgtgatcggcgatgtggtagacatgttcattccttcagtcaacatgtccgtttactttgggtcgaagcacgtcacaaatggctgtgacatcaagccatccattgccatcagccctcctaagctcaccctcaccggcaacatggataacctctactcactggttatgactgatcctgacgcacctagccccagtgaaccaagcatgcgcgagtggatacattggatcttagttgacatacctggaggaacaaacccatttcgcggaaaagagattgtttcatatgtgggaccaagaccacctattggaatacatcgctatatctttgtgttgtttcaacagaaaggacctttaggtcttgtggagcaaccaccaactcgagcaagcttcaacactcgttattttgccaggcaattggacttgggacttccagtggccactgtctacttcaactctcaaaaagaacctgctgttaagaggcgctga

SEQ ID NO: 6 - protein, 190 aa, Glycine max
MRYLSLSTFSLLCITFVVMAASVDPLVVGRVIGDVVDMFIPSVNMSVYFGSKHVTNGCDIKPSIAISPPKLTLTGNMDNLYSLVMTDPDAPSPSEPSMREWIHWILVDIPGGTNPFRGKEIVSYVGPRPPIGIHRYIFVLFQQKGPLGLVEQPPTRASFNTRYFARQLDLGLPVATVYFNSQKEPAVKRR

SEQ ID NO: 7 - DNA, 3468 bp, Glycine max
  source 1..3468: mol_type = genomic DNA
tgtatttgatagtttaattttattttaaaattaaaattcataaataaaataaatgactgcatagtaaataatatataaccaatgcaggaattcaaatactatatgagcaaacaattaaaacttggtatcctctctcctaattttttaattgtatatctaagtctctaatttgagactacaagttaaattagaaaaattttatactaattgatttaggtaaataatggtaattgtctaaataagtaatacaatttataaaataaataagaaaaaaaatgaagtgcatccctagttttatattacaaataatgataaaattgtttattattctacattgtataaaacaataagtttttaaaaaattataaacttaaattcattaaatatacaataaaagaatataatactttatataatgtttttattttcattattattattacgtcacccattttatatgtttttgtgagtataatctgtccaaaaatacattgaatggaaggcaataaatatccacttaaaataatattttttaagacaaaataacatttattttatcttttctcttttttatttatatggattctatatcattttgtgtggatataatttgtctaaaaatataattaatgaatgaaagataataatgatccacttaaaataatattctcaaaggtaacataatttttttatattttattttcttaattaaatagaaatatatattttttgtgtgaatataatttgtttaaaacatattgaatgagaaaccataattaatgatccacttaaaataatattttttaaggtaaaataatattactccagtcacctctaacatattatgaagtatttcttttattttgagagctggggataatatctctctgtcaatacatgcatccaattacctttaacagagtgacctcataagtcatttattttatgtctttaatgagtgttaactttaactccactgaagaatcttccacgtcatccatttatgaaaccctttaaacagccatggccccacctcactatagacaaacaaggaactgtgggagttacgaaaagaggcattgttctcctactgcagaaaccaccattgcaatttgcaccctcattctcacgttacacacactctgcttatattgtagagcaagcatggcttgaagtttgaacccatttactgtttttctgatgcagttagtttcaaatgggcggctttgatatggccatttgtcacaaagaaacagaagatgactgatttactctctgcaaatccaccaacgtgtcatccattgcatgtccctgattttacgatggtccatgcatgatcacaaaattacaaaatagaggtccctgatattttacccttcagtttttttcttttctttttgagctatataaaccctaccctatacactgtaagcaaccaccagaaactctcactagcttagtttctatatatgcgttatttgtctctgagtactttttctctcttgtgcattacctttgtggtcatggcagcctccgtggatcccctagtggttggtcgcgtgatcggcgatgtggtagacatgttcattccttcagtcaacatgtccgtttactttgggtcgaagcacgtcacaaatggctgtgacatcaagccatccattgccatcagccctcctaagctcaccctcaccggcaacatggataacctctacacactggtattcattcatgtttctgttgagagatctcacatgcatgcatcaaggtttttcttaattttatgctttctctttttctttaatgtgcctacaggttatgactgatcctgacgcacctagccccagtgaaccaagcatgcgcgagtggatacattggttagtaaatttgttttagttcttcctattcttagtttctaactccatatatataaactgatatgatcttgctgtttccacttattttccaggatcttagttgacatacctggaggaacaaacccatttcgcggtagtaaaagaaatcttttggtctacttcatatatgacatttttattttcttctatttataaaagtttcatgatgttgcatgcatgagtactactgggttcagttctacttaattaatttttcttttcttcgggctaaaacatcatgttttttaattttttgggcgtccatcgcactgactctcgatttcagaaacatttttcttcactggtcataaaaatcttaggccatttctggcaaaatcaacatgcatgttcctacctaggctcctcgcatgcatgtttcagatttcttctggaaaaaaaaatcacagaatttcagggctatgtcacatcttgtgtgcacactctctttctatacatcaaatggtactctcacacctgtgatctaccaaaatttggatccaatgtccaccatcccccacgtgtcctgcttgtgcctgctatgaacacgtgtcacaacaaatattccatctctaaattctctgaaataatcgttaaataattgttaagtatagtaaataaataaattctcaattattctgtcatattttctctaaaaccattcttcaataaattaattttagattttctccgtgcatgcaccaatttagataacatgtgttagctagggaatctccttttatgagatcatcacacgtgccccatagcttgcatgttatcaaatttcagaattccatcacaagcaaatgtgtcaaacatgaagcaatatttctccaactccaaagtaaaaatattttgaattatcacctttctttggtcctgtcatttctaaaatataatcttcgcatctagcatacaggatattagattaatttttcctactttcaacagttaacattgatttcttgtaaacaataataatttgattcatgcggaacaaagaaatctaataattaatcaagtaaatggtcttctttgcagggaaacaaagaaatctagtaattaatcaagtaaattgtcttctttgcaggaaaagagattgtttcatatgtgggaccaagaccacctattggaatacatcgctatatctttgtgttgtttcaacagaaaggacctttaggtcttgtggagcaaccaccaactcgagcaagcttcaacactcgttattttgccaggcaattggacttgggacttccagtggccactgtctacttcaactctcaaaaagaacctgctgttaagaggcgctgaatctagctatattgtaaccatcagtgtctctcttgagatatgcatggttggaatatacttttaagatatcagaactatataaaatctatgactagtgcgtaaataatgcagggagagggtgtgtgtaaagtaatatagttgtccaagacatgagtggtgccgaccatttccccggctttgataactttttttctctatatatatacctctctttcactctatcaaatatataaagttaatctttatt

SEQ ID NO: 8 - DNA, 402 bp, Glycine max
  source 1..402: mol_type = genomic DNA
attgatttcttgtaaacaataataatttgattcatgcggaacaaagaaatctaataattaatcaagtaaatggtcttctttgcagggaaacaaagaaatctagtaattaatcaagtaaattgtcttctttgcaggaaaagagattgtttcatatgtgggaccaagaccacctattggaatacatcgctatatctttgtgtcctttcaacagaaaggacctttaggtcttgtggagcaaccaccaactcgagcaagcttcaacactcgttattttgccaggcaattggacttgggacttccagtggccactgtctacttcaactctcaaaaagaacctgctgttaagaggcgctgaatctagctatattgtaaccatcagtgtctctcttgagatatgcatgg

SEQ ID NO: 9 - DNA, 20 bp, synthetic construct
  source 1..20: mol_type = other DNA
atatctttgtgtcctttcaa

SEQ ID NO: 10 - DNA, 27 bp, synthetic construct
  source 1..27: mol_type = other DNA
tgcaggaaaagagattgtttcatatgt

SEQ ID NO: 11 - DNA, 20 bp, synthetic construct
  source 1..20: mol_type = other DNA
cttgctcgagttggtggttg

SEQ ID NO: 12 - DNA, 402 bp, Glycine max
  source 1..402: mol_type = genomic DNA
attgatttcttgtaaacaataataatttgattcatgcggaacaaagaaatctaataattaatcaagtaaatggtcttctttgcagggaaacaaagaaatctagtaattaatcaagtaaattgtcttctttgcaggaaaagagattgtttcatatgtgggaccaagaccacctattggaatacatcgctatatctttgtgttgtttcaacagaaaggacctttaggtcttgtggagcaaccaccaactcgagcaagcttcaacactcgttattttgccaggcaattggacttgggacttccagtggccactgtctacttcaactctcaaaaagaacctgctgttaagaggcgctgaatctagctatattgtaaccatcagtgtctctcttgagatatgcatgg

SEQ ID NO: 13 - DNA, 22 bp, synthetic construct
  source 1..22: mol_type = other DNA
ctatatctttgtgttgtttcaa

SEQ ID NO: 14 - DNA, 573 bp, Glycine soja
  source 1..573: mol_type = genomic DNA
atgcgttatttgtctctgagtactttttctctcttgtgcattacctttgtggtcatggcagcctccgtggatcccctagtggttggtcgcgtgatcggcgatgtggtagacatgttcattccttctgtcaacatgtccgtttactttgggtcgaagcacgtcacaaatggctgtgacatcaagccatccatcgccatcagccctcccaagctcaccctcaccggcaacatggataacctctacacactggttatgactgatcctgacgcacctagccccagtgaaccaagcatgcgtgagtggatacattggatcgtagttgacatacctggaggaacaaacccatttcgcggaaaagagattgtttcatatgtgggaccaaggccacctattggaatacatcgctatatctttgtgttgtttcaacagaaaggacctttaggtcttgtggagcaaccaccaactcgtgcaagcttcaacactcgttattttgccaggcaattggacttgggacttccagtggccactgtctacttcaactctcaaaaagaacccgctgttaagaggcgctga

SEQ ID NO: 15 - protein, 190 aa, Glycine soja
MRYLSLSTFSLLCITFVVMAASVDPLVVGRVIGDVVDMFIPSVNMSVYFGSKHVTNGCDIKPSIAISPPKLTLTGNMDNLYTLVMTDPDAPSPSEPSMREWIHWIVVDIPGGTNPFRGKEIVSYVGPRPPIGIHRYIFVLFQQKGPLGLVEQPPTRASFNTRYFARQLDLGLPVATVYFNSQKEPAVKRR

SEQ ID NO: 16 - DNA, 401 bp, Glycine max
  source 1..401: mol_type = genomic DNA
gaaactaagaataggaagaactaaaacaaatttactaaccaatgtatccactcgcgcatgcttggttcactggggctaggtgcgtcaggatcagtcataacctgtaggcacattaaagaaaaagagaaagcataaaattaagaaaaaccttgatgcatgcatgtgagatctctcaacagaaacatgaatgaataccagtgtgtagaggttatccatgttgccggtgagggtgagcttaggagggctgatggcaatggatggcttgatgtcacagccatttgtgacgtgcttcgacccaaagtaaacggacatgttgactgaaggaatgaacatgtctaccacatcgccgatcacgcgaccaaccactaggggatccacggaggctgccatgaccacaaagg

SEQ ID NO: 17 - DNA, 17 bp, synthetic construct
  source 1..17: mol_type = other DNA
tctacacactggtattc

SEQ ID NO: 18 - DNA, 401 bp, Glycine max
  source 1..401: mol_type = genomic DNA
gaaactaagaataggaagaactaaaacaaatttactaaccaatgtatccactcgcgcatgcttggttcactggggctaggtgcgtcaggatcagtcataacctgtaggcacattaaagaaaaagagaaagcataaaattaagaaaaaccttgatgcatgcatgtgagatctctcaacagaaacatgaatgaataccagtgagtagaggttatccatgttgccggtgagggtgagcttaggagggctgatggcaatggatggcttgatgtcacagccatttgtgacgtgcttcgacccaaagtaaacggacatgttgactgaaggaatgaacatgtctaccacatcgccgatcacgcgaccaaccactaggggatccacggaggctgccatgaccacaaagg

SEQ ID NO: 19 - DNA, 18 bp, synthetic construct
  source 1..18: mol_type = other DNA
cctctactcactggtatt

SEQ ID NO: 20 - DNA, 21 bp, synthetic construct
  source 1..21: mol_type = other DNA
accttgatgcatgcatgtgag

SEQ ID NO: 21 - DNA, 20 bp, synthetic construct
  source 1..20: mol_type = other DNA
aagcacgtcacaaatggctg

SEQ ID NO: 22 - protein, 11 aa, synthetic construct
VDPLVVGRVIG

SEQ ID NO: 23 - protein, 10 aa, synthetic construct
MTDPDAPSPS

SEQ ID NO: 24 - protein, 13 aa, synthetic construct
YFNXQKEPXXXRR

SEQ ID NO: 25 - protein, 13 aa, synthetic construct
YFNXQKEPXXXRR

SEQ ID NO: 26 - DNA, 204 bp, Glycine max
  source 1..204: mol_type = genomic DNA
gccatttgtgacgtgcttcgacccaaagtaaacggacatgttgactgaaggaatgaacatgtctaccacatcgccgatcacgcgaccaaccactaggggatccacggaggctgccatgaccacaaaggtaatgcacaagagagaaaaagtactcagagacaaataacgcatatatagaaactaagctagtgagagtttctggtg

SEQ ID NO: 27 - DNA, 13 bp, synthetic construct
  source 1..13: mol_type = other DNA
cctccgtggatcc

SEQ ID NO: 28 - DNA, 200 bp, Glycine max
  source 1..200: mol_type = genomic DNA
gccatttgtgacgtgcttcgacccaaagtaaacggacatgttgactgaaggaatgaacatgtctaccacatcgccgatcacgcgaccaaccactaggggacggaggctgccatgaccacaaaggtaatgcacaagagagaaaaagtactcagagacaaataacgcatatatagaaactaagctagtgagagtttctggtg

SEQ ID NO: 29 - DNA, 15 bp, synthetic construct
  source 1..15: mol_type = other DNA
cctccgtcccctagt

SEQ ID NO: 30 - DNA, 20 bp, synthetic construct
  source 1..20: mol_type = other DNA
catgtctaccacatcgccga

SEQ ID NO: 31 - DNA, 21 bp, synthetic construct
  source 1..21: mol_type = other DNA
caccagaaactctcactagct

SEQ ID NO: 32 - DNA, 201 bp, Glycine max
  source 1..201: mol_type = genomic DNA
atttgtgacgtgcttcgacccaaagtaaacggacatgttgactgaaggaatgaacatgtctaccacatcgccgatcacgcgaccaaccactaggggatccacggaggctgccatgaccacaaaggtaatgcacaagagagaaaaagtactcagagacaaataacgcatatatagaaactaagctagtgagagtttctggtg

SEQ ID NO: 33 - DNA, 13 bp, synthetic construct
  source 1..13: mol_type = other DNA
cctccgtggatcc

SEQ ID NO: 34 - DNA, 200 bp, Glycine max
  source 1..200: mol_type = genomic DNA
atttgtgacgtgcttcgacccaaagtaaacggacatgttgactgaaggaatgaacatgtctaccacatcgccgatcacgcgaccaaccactaggggatcccggaggctgccatgaccacaaaggtaatgcacaagagagaaaaagtactcagagacaaataacgcatatatagaaactaagctagtgagagtttctggtg

SEQ ID NO: 35 - DNA, 12 bp, synthetic construct
  source 1..12: mol_type = other DNA
cctccgggatcc

SEQ ID NO: 36 - DNA, 20 bp, synthetic construct
  source 1..20: mol_type = other DNA
catgtctaccacatcgccga

SEQ ID NO: 37 - DNA, 21 bp, synthetic construct
  source 1..21: mol_type = other DNA
caccagaaactctcactagct

SEQ ID NO: 38 - DNA, 200 bp, Glycine max
  source 1..200: mol_type = genomic DNA
tttgtgacgtgcttcgacccaaagtaaacggacatgttgactgaaggaatgaacatgtctaccacatcgccgatcacgcgaccaaccactaggggatccacggaggctgccatgaccacaaaggtaatgcacaagagagaaaaagtactcagagacaaataacgcatatatagaaactaagctagtgagagtttctggtg

SEQ ID NO: 39 - DNA, 13 bp, synthetic construct
  source 1..13: mol_type = other DNA
cctccgtggatcc

SEQ ID NO: 40 - DNA, 201 bp, Glycine max
  source 1..201: mol_type = genomic DNA
tttgtgacgtgcttcgacccaaagtaaacggacatgttgactgaaggaatgaacatgtctaccacatcgccgatcacgcgaccaaccactaggggatccagcggaggctgccatgaccacaaaggtaatgcacaagagagaaaaagtactcagagacaaataacgcatatatagaaactaagctagtgagagtttctggtg

SEQ ID NO: 41 - DNA, 13 bp, synthetic construct
  source 1..13: mol_type = other DNA
cctccgctggatc

SEQ ID NO: 42 - DNA, 20 bp, synthetic construct
  source 1..20: mol_type = other DNA
catgtctaccacatcgccga

SEQ ID NO: 43 - DNA, 21 bp, synthetic construct
  source 1..21: mol_type = other DNA
caccagaaactctcactagct

SEQ ID NO: 44 - DNA, 51 bp, Glycine max
  source 1..51: mol_type = genomic DNA
cattagtcactgctaaggagatactyctgtattttgctgacagcggaaggt

SEQ ID NO: 45 - DNA, 51 bp, Glycine max
  source 1..51: mol_type = genomic DNA
gcctataataaaagttgagatgattygagtttattactttatttgccattt

SEQ ID NO: 46 - DNA, 51 bp, Glycine max
  source 1..51: mol_type = genomic DNA
cgtcccttgttaggagttaataacawaagaattggggtcaattcgcaactc

SEQ ID NO: 47 - DNA, 51 bp, Glycine max
  source 1..51: mol_type = genomic DNA
aaaattatactagtccccttgatttytccacccgtaaaactatcagtatgg

SEQ ID NO: 48 - DNA, 51 bp, Glycine max
  source 1..51: mol_type = genomic DNA
gaaactgaaggcagtgaaaggcgcastcaaagacgaactcagatcaacaga

SEQ ID NO: 49 - DNA, 51 bp, Glycine max
  source 1..51: mol_type = genomic DNA
gtacccacaacagtaaggataaattsgaaggataaacacatacatatacac

SEQ ID NO: 50 - DNA, 51 bp, Glycine max
  source 1..51: mol_type = genomic DNA
atcaaagccggggaaatggtcggcaycactcatgtcttggacaactatatt

SEQ ID NO: 51 - DNA, 51 bp, Glycine max
  source 1..51: mol_type = genomic DNA
ccggggaaatggtcggcaccactcakgtcttggacaactatattactttac

SEQ ID NO: 52 - DNA, 51 bp, Glycine max
  source 1..51: mol_type = genomic DNA
cacaccctctccctgcattatttackcactagtcatagattttatatagtt

SEQ ID NO: 53 - DNA, 51 bp, Glycine max
  source 1..51: mol_type = genomic DNA
tttatatagttctgatatcttaaaaktatattccaaccatgcatatctcaa

SEQ ID NO: 54 - DNA, 51 bp, Glycine max
  source 1..51: mol_type = genomic DNA
tattccaaccatgcatatctcaagaragacactgatggttacaatatagct

SEQ ID NO: 55 - DNA, 51 bp, Glycine max
  source 1..51: mol_type = genomic DNA
catatctcaagagagacactgatggwtacaatatagctagattcagcgcct

SEQ ID NO: 56 - DNA, 51 bp, Glycine max
  source 1..51: mol_type = genomic DNA
gctagattcagcgcctcttaacagcrggttctttttgagagttgaagtaga

SEQ ID NO: 57 - DNA, 51 bp, Glycine max
  source 1..51: mol_type = genomic DNA
ccaattgcctggcaaaataacgagtrttgaagcttgctcgagttggtggtt

SEQ ID NO: 58 - DNA, 51 bp, Glycine max
  source 1..51: mol_type = genomic DNA
caaaataacgagtgttgaagcttgcwcgagttggtggttgctccacaagac

SEQ ID NO: 59 - DNA, 51 bp, Glycine max
  source 1..51: mol_type = genomic DNA
agtgttgaagcttgctcgagttggtrgttgctccacaagacctaaaggtcc

SEQ ID NO: 60 - DNA, 51 bp, Glycine max
  source 1..51: mol_type = genomic DNA
gagttggtggttgctccacaagaccyaaaggtcctttctgttgaaacaaca

SEQ ID NO: 61 - DNA, 51 bp, Glycine max
  source 1..51: mol_type = genomic DNA
ccacaagacctaaaggtcctttctgytgaaacaacacaaagatatagcgat

SEQ ID NO: 62 - DNA, 51 bp, Glycine max
  source 1..51: mol_type = genomic DNA
aagatatagcgatgtattccaatagktggtcttggtcccacatatgaaaca

SEQ ID NO: 63 - DNA, 51 bp, Glycine max
  source 1..51: mol_type = genomic DNA
tatagcgatgtattccaataggtggycttggtcccacatatgaaacaatct

SEQ ID NO: 64 - DNA, 51 bp, Glycine max
  source 1..51: mol_type = genomic DNA
aataggtggtcttggtcccacatatraaacaatctcttttcctgcaaagaa

SEQ ID NO: 65 - DNA, 51 bp, Glycine max
  source 1..51: mol_type = genomic DNA
tccctgcaaagaagaccatttacttkattaattattagatttctttgttcc

SEQ ID NO: 66 - DNA, 51 bp, Glycine max
  source 1..51: mol_type = genomic DNA
ttattagatttctttgttccgcatgwatcaaattattattgtttacaagaa

SEQ ID NO: 67 - DNA, 51 bp, Glycine max
  source 1..51: mol_type = genomic DNA
attattattgtttacaagaaatcaaygttaactgttgaaagtaggaaaaat

SEQ ID NO: 68 - DNA, 51 bp, Glycine max
  source 1..51: mol_type = genomic DNA
ttgtttacaagaaatcaatgttaacwgttgaaagtaggaaaaattaatcta

SEQ ID NO: 69 - DNA, 51 bp, Glycine max
  source 1..51: mol_type = genomic DNA
tgtttacaagaaatcaatgttaactrttgaaagtaggaaaaattaatctaa

SEQ ID NO: 70 - DNA, 51 bp, Glycine max
  source 1..51: mol_type = genomic DNA
aaagtaggaaaaattaatctaatatmctgtatgctagatgcgaagattata

SEQ ID NO: 71 - DNA, 51 bp, Glycine max
  source 1..51: mol_type = genomic DNA
gtaggaaaaattaatctaatatcctrtatgctagatgcgaagattatattt

SEQ ID NO: 72 - DNA, 51 bp, Glycine max
  source 1..51: mol_type = genomic DNA
aatctaatatcctgtatgctagatgygaagattatattttagaaatgacag

SEQ ID NO: 73 - DNA, 51 bp, Glycine max
  source 1..51: mol_type = genomic DNA
ctagatgcgaagattatattttagamatgacaggaccaaagaaaggtgata

SEQ ID NO: 74 - DNA, 51 bp, Glycine max
  source 1..51: mol_type = genomic DNA
tgcgaagattatattttagaaatgamaggaccaaagaaaggtgataattca

SEQ ID NO: 75 - DNA, 51 bp, Glycine max
  source 1..51: mol_type = genomic DNA
attcaaaatatttttactttggagtyggagaaatattgcttcatgtttgac

SEQ ID NO: 76 - DNA, 51 bp, Glycine max
  source 1..51: mol_type = genomic DNA
ttactttggagttggagaaatattgmttcatgtttgacacatttgcttgtg

SEQ ID NO: 77 - DNA, 51 bp, Glycine max
  source 1..51: mol_type = genomic DNA
aaatattgcttcatgtttgacacatktgcttgtgatggaattctgaaattt

SEQ ID NO: 78 - DNA, 51 bp, Glycine max
  source 1..51: mol_type = genomic DNA
ggcacgtgtgatgatctcataaaagkagattccctagctaacacatgttat

SEQ ID NO: 79 - DNA, 51 bp, Glycine max
  source 1..51: mol_type = genomic DNA
atgttatctaaattggtgcatgcackgagaaaatctaaaattaatttattg

SEQ ID NO: 80 - DNA, 51 bp, Glycine max
  source 1..51: mol_type = genomic DNA
cctgaaattctgtgatttttttttcyagaagaaatctgaaacatgcatgcg

SEQ ID NO: 81 - DNA, 51 bp, Glycine max
  source 1..51: mol_type = genomic DNA
tgtttctgaaatcgagagtcagtgcratggacgcccaaaaaattaaaaaac

SEQ ID NO: 82 - DNA, 51 bp, Glycine max
  source 1..51: mol_type = genomic DNA
gagagtcagtgcgatggacgcccaaraaattaaaaaacatgatgttttagc

SEQ ID NO: 83 - DNA, 51 bp, Glycine max
  source 1..51: mol_type = genomic DNA
tatatgaagtagaccaaaagatttcktttactaccgcgaaatgggtttgtt

SEQ ID NO: 84 - DNA, 51 bp, Glycine max
  source 1..51: mol_type = genomic DNA
tttgttcctccaggtatgtcaactamgatcctggaaaataagtggaaacag

SEQ ID NO: 85 - DNA, 51 bp, Glycine max
  source 1..51: mol_type = genomic DNA
agatcctggaaaataagtggaaacakcaagatcatatcagtttatatatat

SEQ ID NO: 86 - DNA, 51 bp, Glycine max
  source 1..51: mol_type = genomic DNA
caagatcatatcagtttatatatatrgagttagaaactaagaataggaaga

SEQ ID NO: 87 - DNA, 51 bp, Glycine max
  source 1..51: mol_type = genomic DNA
aatttactaaccaatgtatccactcrcgcatgcttggttcactggggctag

SEQ ID NO: 88 - DNA, 51 bp, Glycine max
  source 1..51: mol_type = genomic DNA
gagatctctcaacagaaacatgaatkaataccagtgtgtagaggttatcca

SEQ ID NO: 89 - DNA, 51 bp, Glycine max
  source 1..51: mol_type = genomic DNA
ccatgttgccggtgagggtgagcttrggagggctgatggcaatggatggct

SEQ ID NO: 90 - DNA, 51 bp, Glycine max
  source 1..51: mol_type = genomic DNA
gggtgagcttaggagggctgatggcratggatggcttgatgtcacagccat

SEQ ID NO: 91 - DNA, 51 bp, Glycine max
  source 1..51: mol_type = genomic DNA
tgacgtgcttcgacccaaagtaaacrgacatgttgactgaaggaatgaaca

SEQ ID NO: 92 - DNA, 51 bp, Glycine max
  source 1..51: mol_type = genomic DNA
acccaaagtaaacggacatgttgacwgaaggaatgaacatgtctaccacat

SEQ ID NO: 93 - DNA, 51 bp, Glycine max
  source 1..51: mol_type = genomic DNA
attacaccgtggacgatattataatycaataatatactactgtagctacaa

(19) United States
(12) Patent Application Publication
(10) Pub. No.: US 2026/0206701 A1
(43) Pub. Date: Jul. 23, 2026

(54) METHODS FOR PRODUCING SOYBEAN WITH ALTERED COMPOSITION

(71) Applicant: PIONEER HI-BRED INTERNATIONAL, INC., JOHNSTON, IA (US)

(72) Inventors: JANEL M. BETTIS, URBANDALE, IA (US); JOHN D. EVERARD, GRIMES, IA (US); KRISTIN HAUG COLLET, DES MOINES, IA (US); NICHOLE HUITT, PERRY, IA (US); SIVA S. AMMIRAJU JETTY, JOHNSTON, IA (US); ZHAN-BIN LIU, CLIVE, IA (US); BO SHEN, JOHNSTON, IA (US); SHREEDHARAN SRIRAM, WAUKEE, IA (US); YANG WANG, JOHNSTON, IA (US); GINA MARIE ZASTROW-HAYES, URBANDALE, IA (US)

(73) Assignee: PIONEER HI-BRED INTERNATIONAL, INC., JOHNSTON, IA (US)

(21) Appl. No.: 19/138,749

(22) PCT Filed: Dec. 14, 2023
(86) PCT No.: PCT/US2023/084061

§ 371 Date: Jun. 13, 2025

Related U.S. Application Data
(60) Provisional application No. 63/387,550, filed on Dec. 15, 2022.

(51) Int. Cl. A01H 1/04; A01H 1/00; C07K 14/415; C12Q 1/686; C12Q 1/6895

(52) U.S. Cl. CPC A01H 1/045; A01H 1/104; C07K 14/415; C12Q 1/686; C12Q 1/6895; C12Q 2600/13; C12Q 2600/156

(57) ABSTRACT

The present disclosure provides methods and compositions for producing, detecting and selecting soybean plants and seeds having high seed oil using markers genetically linked to an MFT gene and ingrogressing a high oil MFT allele into soybean plants. The present disclosure also provides soybean plants, plant cells, seed, and grain having increased seed oil content comprising polynucleotides encoding modified MFT polypeptides and methods to increase seed oil content in plants.

### REFERENCE TO SEQUENCE LISTING SUBMITTED ELECTRONICALLY

The official copy of the sequence listing is submitted electronically via Patent Center as an XML formatted sequence listing with a file named 9411_SequenceListing.xml created on Dec. 14, 2022 and having a size of 92,499 bytes and is filed concurrently with the specification. The sequence listing comprised in this XML formatted document is part of the specification and is herein incorporated by reference in its entirety.

### FIELD

This disclosure relates to the field of molecular biology.

### BACKGROUND

Soybean seeds are a source of useful products, such as protein and oil, for human and animal consumption. Thus, generating soybean plants with seeds having increased protein or oil content may contribute to a higher-value crop. However, seed oil content often shows a negative correlation with seed protein content, such that soybeans with increased oil may have reduced protein content.

This disclosure provides compositions and methods to generate and use plants that produce seeds with increased protein and/or oil content. The compositions and methods can be used to develop higher value soybean crops.

### SUMMARY

Provided is a method for producing a soybean plant having high seed oil comprising genotyping a soybean population comprising a plurality of soybean plants or soybean germplasm for the presence of at least one marker genetically linked to a locus comprising or corresponding to an MFT gene having at least 95% identity to SEQ ID NO: 7, the at least one marker detecting a modification in the MFT gene, selecting from the soybean population one or more soybean plants or soybean germplasm comprising the at least one marker, and crossing the selected soybean plant or soybean germplasm with a second soybean plant or soybean germplasm to produce a progeny population, wherein at least one soybean plant or soybean germplasm of the progeny population comprises the at least one marker and has high seed oil as compared to a seed of a control plant. In certain embodiments, the at least one marker comprises an insertion, deletion, polymorphism, or any combination thereof in a coding sequence and/or regulatory sequence of the MFT gene.

Also provided is a method for producing a population of soybean plants or soybean germplasm having an increased seed oil content comprising crossing a first soybean plant or first soybean germplasm comprising a modification at a locus comprising or corresponding to an MFT gene having at least 95% identity to SEQ ID NO: 7, the modification decreasing the expression or activity of an encoded MFT polypeptide, with a second soybean plant or second soybean germplasm to form a soybean plant or soybean germplasm population, genotyping the soybean plant or soybean germplasm population for the presence of at least one marker genetically linked to the locus, the at least one marker detecting the modification, and selecting from the soybean population one or more soybean plants or soybean germplasm comprising the at least one marker. In certain embodiments, the at least one marker comprises an insertion, deletion, polymorphism, or any combination thereof in a coding sequence and/or regulatory sequence of the MFT gene.

Further provided is a method of introgressing a high soybean seed oil MFT allele into a soybean plant comprising crossing a first soybean plant with a second soybean plant to produce a progeny population, the first soybean plant comprising a high soybean seed oil MFT allele comprising a modification of an MFT gene comprising a polynucleotide sequence having at least 95% sequence identity with SEQ ID NO: 7, the modification decreasing the expression or activity of an MFT polypeptide encoded by the MFT gene, genotyping the progeny population for the presence of at least one marker genetically linked to the high soybean seed oil MFT allele, and selecting progeny that comprise the at least one marker to obtain soybean plants comprising the high soybean seed oil MFT allele. In certain embodiments, the modification is polymorphism that decreases expression and/or activity of a polypeptide encoded by the MFT gene compared to a wild-type polypeptide. In certain embodiments, the at least one marker genetically linked to the high oil MFT allele is within 20 centimorgans of the high oil MFT allele.

Provided are soybean cells, soybean plants, and soybean seeds having an increased oil content and comprising a modified MFT gene coding sequence encoding a modified MFT polypeptide comprising an amino acid sequence that is at least 95% identical to SEQ ID NO: 2 and comprises a non-threonine at the amino acid residue corresponding to position 82 of SEQ ID NO: 2. In certain embodiments, the modified MFT polypeptide further comprises a non-leucine at the amino acid residue corresponding to position L140 of SEQ ID NO: 2. In certain embodiments, the oil content of the soybean cell, soybean seed, or seed of the soybean plant is increased by at least a 1 percentage point as compared to a control soybean seed when measured at 13% moisture content. In certain embodiments, the protein content of the soybean cell, soybean seed, or seed of the soybean plant is increased by at least a 0.25 percentage point as compared to a control soybean seed when measured at 13% moisture content.

Also provided is a method of producing the soybean plant having increased oil content and comprising a modified MFT polypeptide sequence comprising introducing into an endogenous MFT gene a modification producing the modified MFT gene coding sequence encoding the modified MFT polypeptide.

Further provided is a method for producing high oil MFT mutant seeds comprising detecting in a mutant seed library the presence of one or more MFT mutant seeds, the one or more MFT mutant seeds comprising a modified MFT gene having at least 95% identity to SEQ ID NO: 7, assaying the seed oil content of the one or more MFT mutant seeds, selecting from the one or more MFT mutant seeds at least one MFT mutant seed having increased seed oil content as compared to a control seed not comprising the modified MFT gene, and crossing a plant grown from the selected MFT mutant seed with a second soybean plant to produce a progeny population, the progeny population comprising at least one plant having increased seed oil content as compared to a seed of a control plant and comprising the modified MFT gene.

### BRIEF DESCRIPTION OF THE DRAWINGS AND THE SEQUENCE LISTING

The disclosure can be more fully understood from the following detailed description and the accompanying drawings and Sequence Listing, which form a part of this application.

FIG. 1 provides a sequence alignment of the MFT amino acid sequences of a wild-type MFT (SEQ ID NO: 2), the HiPO #358 MFT sequence (SEQ ID NO: 4), and the EPHT11 MFT sequence (SEQ ID NO: 6).

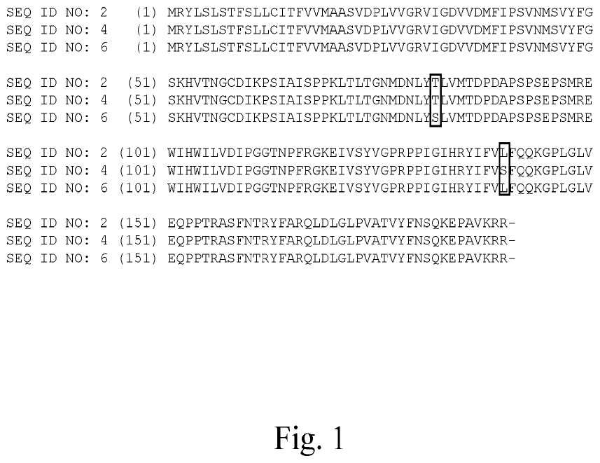

The sequence descriptions (Tables 1A and 1B) summarize the Sequence Listing attached hereto, which is hereby incorporated by reference and complies with the rules governing nucleotide and amino acid sequence disclosures in patent applications as set forth in 37 C.F.R. §§ 1.831-1.835.

TABLE 1A
Sequence Listing Description

| SEQ ID NO: | Name | Organism |
|---|---|---|
| 1 | MFT coding sequence | Glycine max |
| 2 | MFT amino acid sequence | Glycine max |
| 3 | MFT coding sequence HiPO#538 mutant | Glycine max |
| 4 | MFT amino acid sequence HiPO#538 mutant | Glycine max |
| 5 | MFT coding EHPT11 mutant | Glycine max |
| 6 | MFT amino acid sequence EHPT11 mutant | Glycine max |
| 7 | MFT genomic sequence | Glycine max |
| 14 | MFT coding sequence | Glycine soja |
| 15 | MFT amino acid sequence | Glycine soja |
| 22 | MFT amino acid motif 1 | |
| 23 | MFT amino acid motif 2 | |
| 24 | MFT amino acid motif 3 | |
| 25 | MFT amino acid motif 3A | |
| 44-93 | See Table 5 | |

TABLE 1B
Sequence Listing Description - Markers

| | | Wild- | High | High | | |
|---|---|---|---|---|---|---|
| | Wild- | Type | Oil | Oil | | |
| | Type | probe | Allele | Probe | Forward | Reverse |
| | Allele | SEQ | SEQ | SEQ | Primer | Primer |
| | SEQ ID | ID | ID | ID | SEQ ID | SEQ ID |
| Marker Assay | NO: | NO: | NO: | NO: | NO: | NO: |
| S101AY8-00-Q002 | | | 8 | 9 | 10 | 11 |
| High oil | | | | | | |
| S101AY8-00-Q002 | 12 | 13 | | | 10 | 11 |
| wild-type | | | | | | |
| S2000A7-001-Q001 | 16 | 17 | 18 | 19 | 20 | 21 |
| S2000A3-001-Q001 | 26 | 27 | 28 | 29 | 30 | 31 |
| S2000A4-001-Q001 | 32 | 33 | 34 | 35 | 36 | 37 |
| S2000A5-001-Q001 | 38 | 39 | 40 | 41 | 42 | 43 |

### DETAILED DESCRIPTION

The present disclosure provides methods and compositions for producing, detecting, and selecting soybean plants and soybean seeds comprising a modification at the Mother of Flowering Time (MFT) genomic locus on chromosome 5 (glyma.05g244100) that results in a soybean plant producing seeds having an increased oil content and/or increased protein content as compared to a control soybean plant not comprising the modification. In certain embodiments of the methods and compositions described herein the MFT genomic locus comprises a polynucleotide sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 7.

Accordingly, provided is a method for producing a soybean plant or soybean germplasm having high seed oil or increased seed oil comprising genotyping a soybean population comprising a plurality of soybean plants or soybean germplasm to detect the presence of at least one (e.g., 1, 2, 3, 4, 5, 6, 7, 8, 9, 10 or more) marker genetically linked to a genomic locus comprising or corresponding to an MFT gene, the at least one marker detecting a modification in the MFT gene; and selecting from the soybean population one or more soybean plants or soybean germplasm comprising the at least one marker. In certain embodiments, the method comprises detecting two or more markers genetically linked to the locus.

In certain embodiments, the method further comprises crossing the selected soybean plant or soybean germplasm with a second soybean plant or soybean germplasm to produce a progeny population, wherein at least one soybean plant or soybean germplasm of the progeny population comprises the at least one marker and has high seed oil as compared to a seed of a control plant.

In certain embodiments, the seed oil content of the least one soybean plant or soybean germplasm of the progeny population comprising the at least one marker has at least about a 0.1, 0.5, 1.0, 1.5, 2, 2.5, 3, 3.5, 4, 5, 10, or 15 and less than 20, 15, 10, 9, 8, 7, 6, or 5 percentage point increase in total oil measured on a dry weight basis, or seed weight adjusted to 13% moisture, as compared to a control seed (e.g., seed comprising from a plant not comprising the at least one marker). In certain embodiments, the progeny seed further comprises at least a 0.1, 0.5, 1, 1.5, 2, 2.5, 3, 3.5, 4, 4.5, or 5 and less than 15, 14, 13, 12, 11, 10, 9, 8, 7, 6, 5, 4.5, 4, 3.5, 3, 2.5, 2, 1.5, 1, or 0.5 percentage point increase in total protein measured on a dry weight basis, or seed weight adjusted to 13% moisture, as compared to a control seed (e.g., seed comprising from a plant not comprising the at least one marker). As used herein, “percent increase” refers to a change or difference expressed as a fraction of the control value, e.g. {[modified/transgenic/test value (%)−control value (%)]/control value (%)}×100%=percent change., or {[value obtained in a first location (%)−value obtained in second location (%)]/value in the second location (%)}×100=percent change. As used herein, “percentage point” (pp) difference, change, increase or decrease refers to the arithmetic difference of two percentages, e.g. [transgenic or genetically modified value (%)−control value (%)]=percentage points. For example, a modified seed may contain 20% by weight of a component and the corresponding unmodified control seed may contain 15% by weight of that component. The difference in the component between the control and transgenic seed would be expressed as 5 percentage points.

As used herein, “marker” or “molecular marker” “marker loci” or “marker locus” denotes a nucleic acid sequence that is sufficiently unique to characterize a specific locus on the genome.

In certain embodiments, the at least one marker comprises or detects an insertion, deletion, polymorphism (e.g., single nucleotide polymorphism (SNP)), or any combination thereof in a coding sequence and/or regulatory sequence of the MFT gene. In certain embodiments, the at least one marker comprises or detects an insertion of 1, 2, 3, 4, 5, 6, 7, 8, 9, 10, 15, 20, 25, 30, 35, 40, 45, 50, 100 or more nucleotides in the MFT coding and/or regulatory sequence. In certain embodiments, the at least one marker comprises or detects a deletion of 1, 2, 3, 4, 5, 6, 7, 8, 9, 10, 15, 20, 25, 30, 35, 40, 45, 50, 100 or more nucleotides in the MFT coding and/or regulatory sequence. In certain embodiments, the marker comprises or detects a non-synonymous polymorphism in the MFT coding sequence resulting in the encoded MFT polypeptide comprising a modification decreasing the expression, stability and/or activity of the polypeptide. In certain embodiments, the marker comprises or detects a polymorphism in an MFT coding sequence such that the polymorphism results in a coding sequence encoding an MFT polypeptide comprising a non-leucine at the amino acid residue corresponding to position L140 of SEQ ID NO: 2, a non-threonine at the amino acid residue corresponding to position 82 of SEQ ID NO: 2 or a combination thereof.

In certain embodiments, the marker comprises or detects an insertion, deletion, or polymorphism introducing a premature stop codon in an MFT coding sequence resulting in a truncated MFT polypeptide. In certain embodiments of the methods and compositions described herein the MFT coding sequence comprises a polynucleotide sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 1.

In certain embodiments, the at least one marker comprises or detects an insertion, deletion, polymorphism, or any combination thereof in an MFT promoter sequence (e.g., nucleotides 1-1431 of SEQ ID NO: 7), a 5′-UTR (e.g., nucleotides 1432-1469 of SEQ ID NO: 7), an intron (e.g., nucleotides 1719-1812, 1875-1966, and 2008-3000 of SEQ ID NO: 7), or a 3′-UTR (e.g., nucleotides 3222-3468 of SEQ ID NO: 7), or any combination thereof. In certain embodiments, the at least one marker comprises or detects an insertion of 1, 2, 3, 4, 5, 6, 7, 8, 9, 10, 15, 20, 25, 30, 35, 40, 45, 50, 100 or more and, for example, less than 10,000, 5,000, 2,000, 1,000, 500, 200, or 100 nucleotides in the MFT regulatory sequence. In certain embodiments, the at least one marker comprises or detects a deletion of 1, 2, 3, 4, 5, 6, 7, 8, 9, 10, 15, 20, 25, 30, 35, 40, 45, 50, 100 or more and, for example, less than 5,000, 4,000, 3,500, 3,000, 2,500, 2,000, 1,500, 1,000, 500, 200, or 100 nucleotides in the MFT regulatory sequence. In certain embodiments, the modification in the MFT regulatory sequence results in decreased expression of the encoded MFT polypeptide. In certain embodiments, the at least one marker comprises or detects a modification (e.g., insertion, deletion, polymorphism) in the MFT promoter sequence. In certain embodiments, the modification in the MFT promoter sequence results in decreased expression of the encoded MFT polypeptide.

A “regulatory sequence” generally refers to a transcriptional regulatory element involved in regulating the transcription of a nucleic acid molecule such as a gene or a target gene. The regulatory element is a nucleic acid and may include a promoter, an enhancer, an intron, a 5′-untranslated region (5′-UTR, also known as a leader sequence), or a 3′-UTR or a combination thereof. A “promoter” refers to a region of DNA upstream from the start of transcription and involved in recognition and binding of RNA polymerase and other proteins to initiate transcription. An “enhancer” element is any nucleic acid molecule that increases transcription of a nucleic acid molecule when functionally linked to a promoter regardless of its relative position. An “intron” is an intervening sequence in a gene that is transcribed into RNA but is then excised in the process of generating the mature mRNA. The term is also used for the excised RNA sequences. The 5′ untranslated region (5′UTR) (also known as a translational leader sequence or leader RNA) is the region of an mRNA that is directly upstream from the initiation codon. This region is involved in the regulation of translation of a transcript by differing mechanisms in viruses, prokaryotes and eukaryotes. The “3′ non-coding sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor.

In certain embodiments, the at least one marker comprises or detects a deletion of all or part of the MFT gene or MFT coding sequences such that the at least one marker is genetically linked to a locus corresponding to the MFT gene or MFT coding sequences, such as those found in flanking regions of the MFT gene.

In certain embodiments, the at least one marker genetically linked to the locus is selected from the group consisting of a CC at marker S101AY8-00-Q002, a T at marker S2000A7-001-Q001, a 4 base pair deletion at marker S2000A3-001-Q001, a 1 base pair deletion at marker S2000A4-001-Q001, a G insertion at marker S2000A5-001-Q001, and combinations thereof.

As used herein a “deletion,” “deletion mutation,” “deletion modification” or the like, refers to a mutation in which the indicated nucleotide or nucleotides is removed from the polynucleotide sequence, so that, when aligned to the reference sequence (e.g., SEQ ID NO: 7) the mutated sequence does not have a nucleotide corresponding to the indicated position of the reference sequence. An “insertion,” “insertion mutation,” “insertion modification,” or the like, refers to a mutation in which at least one nucleotide is added to the polynucleotide sequence, so that, when aligned to the reference sequence (e.g., SEQ ID NO: 7) the mutated sequence contains an additional nucleotide corresponding to the indicated position or region of the reference sequence.

A “polymorphism,” “nucleotide substitution,” or the like, refers to a mutation or modification in which the indicated nucleotide residue is replaced with a different nucleotide, so that, when aligned to the reference sequence (e.g., SEQ ID NO: 7) the mutated or modified sequence does not have the same nucleotide at the indicated position. The polymorphism may be present in a gene coding region or in a regulatory region. As used herein, a polymorphism in a gene coding sequence that results in a mutation or modification in the encoded polypeptide is considered be a non-synonymous mutation or modification. The non-synonymous mutation or modification may result in the encoded polypeptide having a substitution mutation or modification or a truncation (e.g., premature stop codon).

An “amino acid substitution,” “substitution mutation,” or the like, refers to a mutation in which the indicated amino acid residue is replaced with a different amino acid residue, so that, when aligned to the reference sequence (e.g., SEQ ID NO: 2) the mutated sequence does not have the same amino acid at the indicated position.

As used herein, a “modification” “mutation” or the like refers a polynucleotide or polypeptide that has been altered. Such that a “mutated polynucleotide” or “mutated polypeptide” has a sequence that differs from the sequence of the corresponding non-mutated polynucleotide or polypeptide by at least one nucleotide or amino acid. In certain embodiments of the disclosure, the mutated polynucleotide or polynucleotide comprises an alteration that results from a guide polynucleotide/Cas endonuclease system as disclosed herein. A mutated or modified plant is a plant comprising a mutated polynucleotide or polypeptide.

In certain embodiments, the presence of the at least one marker is detected using a suitable amplification-based detection method, such as, for example, PCR, RT-PCR, and LCR. PCR, RT-PCR, and LCR can be used as amplification and amplification-detection methods for amplifying nucleic acids of interest (e.g., those comprising marker loci), facilitating detection of the markers. Such nucleic acid amplification techniques can be used in the methods to amplify and/or detect nucleic acids of interest, such as nucleic acids comprising marker loci. In these types of methods, nucleic acid primers may be hybridized to the conserved regions flanking the polymorphic marker region. In certain methods, nucleic acid probes that bind to the amplified region can be also employed. In general, synthetic methods for making oligonucleotides, including primers and probes, are well known in the art. The primers and probes for use in the methods described herein are not particularly limited and may be designed using methods and/or software known in the art, such as, for example, LASERGENE® (bioinformatics software for molecular biology) or Primer3. It is not intended that the primers be limited to generating an amplicon of any particular size. For example, the primers used to amplify the markers herein are not limited to amplifying the entire region of the relevant locus. In some embodiments, marker amplification produces an amplicon at least 20 nucleotides in length, or alternatively, at least 50 nucleotides in length, or alternatively, at least 100 nucleotides in length, or alternatively, at least 200 nucleotides in length.

Non-limiting examples of polynucleotide primers useful for detecting the high oil or high protein markers provided herein are provided in Table 1B and include, for example, SEQ ID NOs: 10, 11, 20, 21, 30, 31, 36, 37, 42, and/or 43 or variants or fragments thereof.

Non-limiting examples of polynucleotide probes useful for detecting the high oil or high protein markers provided herein are provided in Table 1B and include, for example, SEQ ID NO: 9, 19, 29, 35 and 41 or any combination thereof.

In certain embodiments, probes used in methods disclosed herein such as for detecting the markers described herein will possess a detectable label. Any suitable label can be used with a probe. Detectable labels suitable for use with nucleic acid probes include, for example, any composition detectable by spectroscopic, radioisotopic, photochemical, biochemical, immunochemical, electrical, optical, or chemical means. Useful labels include biotin for staining with labeled streptavidin conjugate, magnetic beads, fluorescent dyes, radiolabels, enzymes, and colorimetric labels. Other labels include ligands, which bind to antibodies labeled with fluorophores, chemiluminescent agents, and enzymes. Detectable labels may also include reporter-quencher pairs, such as are employed in Molecular Beacon and TaqMan™ probes. Generally, whether the quencher is fluorescent or simply releases the transferred energy from the reporter by non-radiative decay, the absorption band of the quencher should at least substantially overlap the fluorescent emission band of the reporter to optimize the quenching. Non-fluorescent quenchers or dark quenchers typically function by absorbing energy from excited reporters, but do not release the energy radiatively. Selection of appropriate reporter-quencher pairs for particular probes may be undertaken in accordance with known techniques.

Further, it will be appreciated that amplification is not a requirement for marker detection—for example, one can directly detect unamplified genomic DNA simply by performing a Southern blot on a sample of genomic DNA. Procedures for performing Southern blotting, amplification e.g., (PCR, LCR, or the like), and many other nucleic acid detection methods are well established.

Real-time amplification assays, including MB or TaqMan™ based assays, are especially useful for detecting polymorphisms such as SNPs. In such cases, the methods can include a step of designing a probe to bind to the amplicon region that includes the polymorphic locus, with one allele-specific probe being designed for each possible polymorphic allele. For instance, if there are two known alleles for a particular polymorphic locus, “A” or “C,” then one probe is designed with an “A” at the polymorphic position, while a separate probe is designed with a “C” at the polymorphic position. While the probes are typically identical to one another other than at the polymorphic position or position, they need not be. For instance, the two allele-specific probes could be shifted upstream or downstream relative to one another by one or more bases. However, if the probes are not otherwise identical, they should be designed such that they bind with approximately equal efficiencies, which can be accomplished by designing under a strict set of parameters that restrict the chemical properties of the probes. Further, a different detectable label, for instance a different reporter-quencher pair, is typically employed on each different allele-specific probe to permit differential detection of each probe. In certain examples, each allele-specific probe for a certain polymorphic locus is 11-20 nucleotides in length, dual-labeled with a florescence quencher at the 3′ end and either the 6-FAM (6-carboxyfluorescein) or VIC (4,7,2′-trichloro-7′-phenyl-6-carboxyfluorescein) fluorophore at the 5′ end.

To effectuate polymorphism detection, a real-time PCR reaction can be performed using primers that amplify the region including the polymorphic locus, for instance the sequences listed in Tables 1B and 5, the reaction being performed in the presence of all allele-specific probes for the given polymorphic locus. By then detecting signal for each detectable label employed and determining which detectable label(s) demonstrated an increased signal, a determination can be made of which allele-specific probe(s) bound to the amplicon and, thus, which polymorphic allele(s) the amplicon possessed. For instance, when 6-FAM- and VIC-labeled probes are employed, the distinct emission wavelengths of 6-FAM (518 nm) and VIC (554 nm) can be captured. A sample that is homozygous for one allele will have fluorescence from only the respective 6-FAM or VIC fluorophore, while a sample that is heterozygous at the analyzed locus will have both 6-FAM and VIC fluorescence.

Other techniques for detecting polymorphisms can also be employed, such as allele specific hybridization (ASH). ASH technology is based on the stable annealing of a short, single-stranded, oligonucleotide probe to a completely complementary single-stranded target nucleic acid. Detection is via an isotopic or non-isotopic label attached to the probe. For each polymorphism, two or more different ASH probes are designed to have identical DNA sequences except at the polymorphic nucleotides. Each probe will have exact homology with one allele sequence so that the range of probes can distinguish all the known alternative allele sequences. Each probe is hybridized to the target DNA. With appropriate probe design and hybridization conditions, a single-base mismatch between the probe and target DNA will prevent hybridization.

In certain embodiments, the presence of the at least one marker is detected by DNA sequencing. Several methods are available for sequencing, including, but not limited to, hybridization, primer extension, oligonucleotide ligation, nuclease cleavage, minisequencing, and coded spheres. The KASPar® (homogeneous fluorescent genotyping system) and Illumina® Detection Systems (genotyping array system) are additional examples of commercially-available marker detection systems. KASPar® is a homogeneous fluorescent genotyping system which utilizes allele specific hybridization and a unique form of allele specific PCR (primer extension) in order to identify genetic markers (e.g., a particular SNP marker genetically linked to high soybean seed oil content). Illumina® detection systems utilize similar technology such as in a fixed platform format. The fixed platform utilizes a physical plate that can be created with up to, for example, 384 markers. The Illumina® system can be created with a single set of markers and utilize dyes to indicate marker detection.

The systems and methods described herein represent a wide variety of available detection methods which can be utilized to genotype for and detect the presence of the markers described herein (e.g., markers genetically linked to a locus comprising or corresponding to an MFT gene), but any other suitable method could also be used.

As used herein, the term “germplasm” refers to genetic material of or from an individual (e.g., a plant), a group of individuals (e.g., a plant line, variety or family), or a clone derived from a line, variety, species, or culture, or more generally, all individuals within a species or for several species (e.g., maize germplasm collection or Andean germplasm collection). The germplasm can be part of an organism, cell, or can be separate from the organism or cell. In general, germplasm provides genetic material with a specific molecular makeup that provides a physical foundation for some or all of the hereditary qualities of an organism or cell culture. As used herein, germplasm includes cells, seed or tissues from which new plants may be grown, or plant parts, such as leaves, stems, pollen, or cells, that can be cultured into a whole plant.

As used herein, the term “plant” includes plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like.

Also provided herein are methods for producing a population of soybean plants or soybean germplasm having an increased seed oil and/or protein content comprising crossing a first soybean plant or first soybean germplasm comprising a modification at a locus comprising or corresponding to an MFT gene having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 7, the modification decreasing the expression, stability or activity of an encoded MFT polypeptide, with a second soybean plant or second soybean germplasm to form a soybean plant or soybean germplasm population, genotyping the soybean plant or soybean germplasm population for the presence of at least one (e.g., 1, 2, 3, 4, 5, 6, 7, 8, 9, 10 or more) marker genetically linked to the locus, the at least one marker detecting the modification, and selecting from the soybean population one or more soybean plants or soybean germplasm comprising the at least one marker. The at least one marker genetically linked to the locus may be any marker provided herein such as, for example, an insertion, deletion, polymorphism, in a coding sequence of the MFT gene, an insertion, deletion, polymorphism, in a coding sequence of the MFT gene, or any combination thereof. In certain embodiments, the marker is selected from the group consisting of a CC at marker S101AY8-00-Q002, a T at marker S2000A7-001-Q001, a 4 base pair deletion at marker S2000A3-001-Q001, a 1 base pair deletion at marker S2000A4-001-Q001, and a G insertion at marker S2000A5-001-Q001. In certain embodiments, the method comprises detecting two or more markers genetically linked to the locus. The method for genotyping for the presence (i.e., detecting) the marker may be any method described herein or known in the art.

As used herein, the term “crossing”, “crossed”, “cross” or the like refers to a sexual cross and involved the fusion of two haploid gametes via pollination to produce diploid progeny (e.g., cells, seeds or plants). The term encompasses both the pollination of one plant by another and selfing (or self-pollination, e.g., when the pollen and ovule are from the same plant).

In certain embodiments, the seed oil content of the soybean plant or soybean germplasm selected from population comprising the at least one marker has at least about a 0.1, 1.5, 2, 2.5%, 3, 3.5, 4, 5, 10, or 15 and less than 20, 15, 10, 9, 8, 7, 6, or 5 percentage point increase in total oil measured on a dry weight basis, or adjusted to 13% moisture, as compared to a control seed (e.g., seed comprising from a plant not comprising the at least one marker). In certain embodiments, the seed further comprises at least a 0.1, 0.5, 1, 1.5, 2, 2.5, 3, 3.5, 4, 4.5, or 5 and less than 15, 14, 13, 12, 11, 10, 9, 8, 7, 6, 5, 4.5, 4, 3.5, 3, 2.5, 2, 1.5, 1, or 0.5 percentage point increase in total protein measured on a dry weight basis, or adjusted to 13% moisture, as compared to a control seed (e.g., seed comprising from a plant not comprising the at least one marker).

In certain embodiments of the methods described herein, the first soybean plant or soybean germplasm, the second soybean plant or soybean germplasm, or both the first and second soybean plant or soybean germplasm are elite soybean lines. In certain embodiments of the methods described herein, the first soybean plant or soybean germplasm or the second soybean plant or soybean germplasm is an exotic soybean line.

As used herein, and “elite line” is an agronomically superior line that has resulted from many cycles of breeding and selection for superior agronomic performance. Numerous elite lines are available and known to those of skill in the art of soybean breeding. As used herein, an “exotic soybean line” is a strain or germplasm derived from a soybean not belonging to an available elite soybean line or strain of germplasm. In the context of a cross between two soybean plants or strains of germplasm, an exotic germplasm is not closely related by descent to the elite germplasm with which it is crossed. Most commonly, the exotic germplasm is not derived from any known elite line of soybean, but rather is selected to introduce novel genetic elements (typically novel alleles) into a breeding program.

Further provided herein are methods of applying plant breeding techniques to plants and seeds in the disclosed methods, such as introgressing a high soybean seed oil MFT allele from a plant containing the high soybean seed oil MFT allele into a plant that does not contain the allele. In certain embodiments, the methods include crossing a first soybean plant with a second soybean plant to produce a progeny population, the first soybean plant comprising a high soybean seed oil MFT allele comprising a modification of an MFT gene comprising a polynucleotide sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity with SEQ ID NO: 7, the modification decreasing the expression or activity of an MFT polypeptide encoded by the MFT gene, genotyping the progeny population for the presence of at least one marker genetically linked to the high soybean seed oil MFT allele, and selecting progeny that comprise the at least one marker to obtain soybean plants comprising the high soybean seed oil MFT allele. Selected progeny in the methods disclosed herein can be separated from progeny that do not carry the desired trait. In the methods described herein, selected or separated progeny such as following detection of the trait can be grown and have applied to them plant breeding techniques to develop further progeny plants. Plant breeding techniques known in the art and used in a soybean plant breeding program and the methods disclosed herein include, but are not limited to, recurrent selection, mass selection, bulk selection, backcrossing, pedigree breeding, open pollination breeding, restriction fragment length polymorphism enhanced selection, genetic marker enhanced selection, making double haploids, transformation, mutation breeding and genome editing. Often combinations of these techniques are used.

In certain embodiments, the modification comprises an insertion, deletion, or polymorphism of the MFT gene sequence that decreases the expression of an MFT polypeptide encoded by the MFT gene as compared to expression of a control MFT polypeptide (e.g., wild-type MFT polypeptide, SEQ ID NO: 2). In certain embodiments, the modification is an insertion, deletion, or polymorphism of the MFT gene sequence that decreases activity of an MFT polypeptide encoded by the MFT gene as compared to the activity of a control MFT polypeptide (e.g., a wild-type MFT polypeptide, SEQ ID NO: 2). In certain embodiments, the modification decreasing the expression, activity, or both expression and activity is an insertion, deletion or polymorphism that introduces a non-synonymous mutation in the coding sequence of the MFT gene, such as for example, a mutation introducing a premature stop codon, a mutation resulting in the encoded MFT polypeptide comprising a non-leucine at residue L140 of SEQ ID NO:2, a mutation resulting in the encoded MFT polypeptide comprising a non-threonine at residue T82 of SEQ ID NO:2, or a combination thereof. In certain embodiments, the modification decreasing the expression, activity, or both expression and activity is a polymorphism in a regulatory sequence of the MFT gene. In certain embodiments, the modification decreasing the expression, activity, or both expression and activity is an insertion of 1, 2, 3, 4, 5, 6, 7, 8, 9, 10, 15, 20, 25, 30, 35, 40, 45, 50, 100 or more nucleotides in the MFT gene regulatory sequence. In certain embodiments, the modification decreasing the expression, activity, or both expression and activity is a deletion of 1, 2, 3, 4, 5, 6, 7, 8, 9, 10, 15, 20, 25, 30, 35, 40, 45, 50, 100 or more nucleotides in the MFT gene regulatory sequence. In certain embodiments, the modification decreasing the expression, activity, or both expression and activity is a deletion of an MFT gene regulatory sequence, a deletion of an MFT gene coding sequence, or a deletion of the MFT gene sequence.

As used herein, “decreasing expression”, “decreased expression” or the like refers to any detectable reduction in the level of the transcribed polynucleotide or encoded polypeptide as compared to a control plant (e.g., non-modified plant). The level of polynucleotide expression can be measure using routine methods known in the art such as, for example, RT-PCT. The level of polypeptide expression can be measured using routine methods known in the art such as, for example, Western blotting, mass spectrometry, and ELISA.

As used herein, “decrease in activity” “decreased activity” “decreasing activity” and the like refers to any detectable reduction in the function of the polypeptide. The decrease in activity can be any MFT activity known in the art including, but not limited to, changes in expression or activity of downstream polypeptides, MFT polypeptide turnover rate (e.g., polypeptide stability), MFT polypeptide binding (e.g., protein-protein interaction), or MFT polypeptide folding. In certain embodiments, the decreased activity refers to a decrease in the stability of the encoded MFT polypeptide. The decrease in stability may be determined using any method known in the art such as for example, measuring polypeptide turnover or half-life.

“Introgressing”, “introgression” and the like, as used herein, refers to the transmission of a desired allele of a genetic locus from one genetic background to another. For example, introgression of a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele can be, e.g., detected by a marker that is associated with a phenotype, at a QTL, a transgene, or the like. Offspring comprising the desired allele may be repeatedly backcrossed to a line having a desired genetic background and selected for the desired allele, to result in the allele becoming fixed in a selected genetic background. The process of “introgressing” is often referred to as “backcrossing” when the process is repeated two or more times.

As used herein “allele” refers to any of one or more alternative forms of a genetic sequence. In a diploid cell or organism, the two alleles of a given sequence typically occupy corresponding loci on a pair of homologous chromosomes. With regard to a polymorphism marker, allele refers to the specific nucleotide base or bases present at that polymorphic locus in that individual plant. A “high soybean seed oil MFT allele” as used herein refers to an allele at an MFT genomic locus comprising a modification that results in plants having seeds with increased oil content and/or increased protein content as compared to plants not comprising the modification.

In certain embodiments, the soybean plants selected comprising the high soybean seed oil MFT allele have at least about a 0.1, 1.5, 2, 2.5, 3, 3.5, 4, 5, 10, or 15 and less than 20, 15, 10, 9, 8, 7, 6, or 5 percentage point increase in total seed oil measured on a dry weight basis, or adjusted to 13% moisture, as compared to a control seed (e.g., seed comprising from a plant not comprising the at least one marker). In certain embodiments, the seeds of the plants further comprises at least a 0.1, 0.5, 1, 1.5, 2, 2.5, 3, 3.5, 4, 4.5, or 5 and less than 15, 14, 13, 12, 11, 10, 9, 8, 7, 6, 5, 4.5, 4, 3.5, 3, 2.5, 2, 1.5, 1, or 0.5 percentage point increase in total protein measured on a dry weight basis, or adjusted to 13% moisture, as compared to a control seed (e.g., seed comprising from a plant not comprising the at least one marker).

In certain embodiments, the marker genetically linked to the high oil MFT allele is within 50 cM, 40 cM, 30 CM, 25 CM, 20 cM, 15 CM, 10 cM, 9 cM, 8 CM, 7 cM, 6 cM, 5 CM, 4 cM, 3 cM, 2 cM, 1 cM centimorgans (CM) of the high oil MFT allele. In certain embodiments, the marker genetically linked to the high oil MFT allele is within about 1 kb, 2 kb, 3 kb, 4 kb, 5 kb, 6 kb, 7 kb, 8 kb, 9 kb, 10 kb, 11 kb, 12 kb, 13 kb, 14 kb, 15 kb, 16 kb, 17 kb, 18 kb, 19 kb, 20 kb, 21 kb, 22 kb, 23 kb, 24 kb, 25 kb, 26 kb, 27 kb, 28 kb, 29 kb, 30 kb, 35 kb, 40 kb, 45 kb, 50 kb, 55 kb, 60 kb, 65 kb, 70 kb, 75 kb, 80 kb, 85 kb, 90 kb, 95 kb, 100 kb, 110 kb, 120 kb, 130 kb, 140 kb, 150 kb, 160 kb, 170 kb, 180 kb, 190 kb, or about 200 kb of the high oil MFT allele. In certain embodiments, the marker genetically linked to the allele occurs in the region defined by and including in flanking markers SEQ ID NO: 44, 45, 46, 47 or 48 and SEQ ID NO: 93.

A cM is a unit of measure of genetic recombination frequency. One cM is equal to a 1% chance that a trait at one genetic locus will be separated from a trait at another locus due to crossing over in a single generation (meaning the traits segregate together 99% of the time). Because chromosomal distance is approximately proportional to the frequency of crossing over events between traits, there is an approximate physical distance that correlates with recombination frequency. Marker loci are themselves traits and can be assessed according to standard linkage analysis by tracking the marker loci during segregation. Thus, one cM is equal to a 1% chance that a marker locus will be separated from another locus, due to crossing over in a single generation. When a marker is stated to be genetically linked to an allele (e.g., high oil MFT allele) or locus (e.g., locus comprising or corresponding to an MFT gene) it will be understood that the allele or locus generally co-segregates with the marker.

In certain embodiments, the at least one marker genetically linked to the high soybean seed oil MFT allele is selected from the group consisting of a CC at marker S101AY8-00-Q002, a T at marker S2000A7-001-Q001, a 4 base pair deletion at marker S2000A3-001-Q001, a 1 base pair deletion at marker S2000A4-001-Q001, a G insertion at marker S2000A5-001-Q001, or a high oil allele at the indicated position in Table 5.

Also provided are soybean plants, plant cells, plant parts, seeds, and grain comprising a modified MFT gene coding sequence that encodes a modified MFT polypeptide having decreased expression or decreased activity as compared to a non-modified MFT polypeptide (e.g., wild-type MFT polypeptide). In certain embodiments, the modified MFT polypeptide comprises an amino acid sequence that is at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 2 and comprises a non-threonine at the amino acid residue corresponding to position 82 of SEQ ID NO: 2. In certain embodiments, the modified MFT polypeptide sequence comprises a glycine, asparagine, glutamine, alanine, serine, or cysteine at the amino acid residue corresponding to position 82 of SEQ ID NO: 2. In certain embodiments, the modified MFT polypeptide comprises an amino acid sequence that is at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 2 and comprises, a non-leucine at the amino acid residue corresponding to position L140 of SEQ ID NO: 2. In certain embodiments, the modified MFT polypeptide sequence comprises a glycine, asparagine, glutamine, alanine, serine, cysteine, or threonine at the amino acid residue corresponding to position L140 of SEQ ID NO: 2. In certain embodiments, the modified MFT polypeptide comprises an amino acid sequence that is at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 2 and comprises a non-threonine at position at the amino acid residue corresponding to position 82 of SEQ ID NO: 2 and a non-leucine at the amino acid residue corresponding to position L140 of SEQ ID NO: 2. In certain embodiments, the modified MFT polypeptide comprises a glycine, asparagine, glutamine, alanine, serine, or cysteine at the amino acid residue corresponding to position 82 of SEQ ID NO: 2 and a glycine, asparagine, glutamine, alanine, serine, cysteine, or threonine at the amino acid residue corresponding to position L140 of SEQ ID NO: 2. In certain embodiments, the modified MFT polypeptides further comprise at least one amino acid motif selected from the group consisting of VDPLVVGRVIG (SEQ ID NO: 22), MTDPDAPSPS (SEQ ID NO: 23), and YFNX₁QKEPX₂X₃X₄RR (SEQ ID NO: 24), where X is any amino acid. In certain embodiments, the modified MFT polypeptides further comprise each of the amino acid motifs VDPLVVGRVIG (SEQ ID NO: 22), MTDPDAPSPS (SEQ ID NO: 23), and YFNX₁QKEPX₂X₃X₄RR (SEQ ID NO: 24), where X is any amino acid. In certain embodiments, X₁ is S or A, X₂ is A or V, X₃ is V, S, or N, and X₄ is K or R. In certain embodiments, the amino acid motif VDPLVVGRVIG (SEQ ID NO: 22) is present from amino acid positions 23 to 33 corresponding to SEQ ID NO: 2. In certain embodiments, the amino acid motif MTDPDAPSPS (SEQ ID NO: 23) is present from amino acid positions 85 to 94 corresponding to SEQ ID NO: 2. In certain embodiments, the amino acid motif YFNX₁QKEPX₂X₃X₄RR (SEQ ID NO: 24) is present from amino acid positions 178 to 190 corresponding to SEQ ID NO: 2.

As used herein “encoding,” “encoded,” or the like, with respect to a specified nucleic acid, is meant comprising the information for translation into the specified protein. A nucleic acid encoding a protein may comprise non-translated sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code. However, variants of the universal code, such as is present in some plant, animal and fungal mitochondria, the bacterium Mycoplasma capricolum (Yamao, et al., (1985) Proc. Natl. Acad. Sci. USA 82:2306-9) or the ciliate Macronucleus, may be used when the nucleic acid is expressed using these organisms.

The terms “polypeptide,” “peptide” and “protein” are used interchangeably herein to refer to a polymer of amino acid residues. The terms apply to amino acid polymers in which one or more amino acid residues is an artificial chemical analogue of a corresponding naturally occurring amino acid, as well as to naturally occurring amino acid polymers.

As used herein “percent (%) sequence identity” with respect to a reference sequence (subject) is determined as the percentage of amino acid residues or nucleotides in a candidate sequence (query) that are identical with the respective amino acid residues or nucleotides in the reference sequence, after aligning the sequences and introducing gaps, if necessary, to achieve the maximum percent sequence identity, and not considering any amino acid conservative substitutions as part of the sequence identity. Alignment for purposes of determining percent sequence identity can be achieved in various ways that are within the skill in the art, for instance, using publicly available computer software such as BLAST, BLAST-2. Those skilled in the art can determine appropriate parameters for aligning sequences, including any algorithms needed to achieve maximal alignment over the full length of the sequences being compared. The percent identity between the two sequences is a function of the number of identical positions shared by the sequences (e.g., percent identity of query sequence=number of identical positions between query and subject sequences/total number of positions of query sequence×100).

Unless otherwise stated, sequence identity/similarity values provided herein refer to the value obtained using the BLAST 2.0 suite of programs using default parameters (Altschul, et al., (1997) Nucleic Acids Res. 25:3389-402).

In certain embodiments, the seeds of the plant, plant cells, and seeds comprising the modified MFT gene coding sequence that encodes the modified MFT polypeptides have an increase in total oil content when compared to a seed, cell, or plant comprising a comparable polynucleotide which lacks the modification.

In certain embodiments, the oil content in the seeds of the plant, plant cells, and seeds comprising the modified MFT gene coding sequence that encodes the modified MFT polypeptides disclosed herein comprises an increase of at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45% or 50% relative to the oil content measured on a dry weight basis, or adjusted to 13% moisture, of a control seed (e.g., seed expressing the polypeptide without the modifications). In certain embodiments, the oil content in the seeds of the plant, plant cells, and seeds comprising the modified MFT gene coding sequence that encodes the modified MFT polypeptide disclosed herein comprises at least about a 0.1, 1.5, 2, 2.5, 3, 3.5, 4, 5, 10, or 15 and less than 20, 15, 10, 9, 8, 7, 6, or 5 percentage point increase in total oil measured on a dry weight basis, or adjusted to 13% moisture, as compared to a control seed (e.g., seed comprising a non-modified polypeptide).

In certain embodiments, the seeds of the plant, plant cells, and seeds comprising the modified MFT gene coding sequence that encodes the modified MFT polypeptide have an increase in total protein content when compared to a seed or plant comprising a comparable polynucleotide which lacks the modification.

In certain embodiments, the protein content in the seeds of the plant, plant cells, and seeds comprising the modified MFT gene coding sequence that encodes the modified MFT polypeptides disclosed herein comprises an increase of at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45% or 50% relative to the protein content measured on a dry weight basis, or adjusted to 13% moisture, of a control seed (e.g., seed expressing the polypeptide without the modifications). In certain embodiments, the protein content in the in the seeds of the plant, plant cells, and seeds comprising the modified MFT gene coding sequence that encodes the modified MFT polypeptides disclosed herein comprises at least about a 0.1, 0.5, 1, 1.5, 2, 2.5, 3, 3.5, 4, 4.5, or 5 and less than 15, 14, 13, 12, 11, 10, 9, 8, 7, 6, 5, 4.5, 4, 3.5, 3, 2.5, 2, 1.5, 1, or 0.5 percentage point increase in total protein measured on a dry weight basis, or adjusted to 13% moisture, as compared to a control seed (e.g., seed comprising a non-modified polypeptide).

In certain embodiments, the seeds of the plant, plant cells, and seeds comprising the modified MFT gene coding sequence that encodes the modified MFT polypeptide have an increase in both total protein and total oil content when compared to a control seed or plant (e.g., a seed or plant comprising a comparable polynucleotide which lacks the modification). The increase in total oil content and total protein content can be any increase described herein.

In certain embodiments, the seeds of the plant, plant cells, and seeds comprising the modified MFT gene coding sequence that encodes the modified MFT polypeptide have modified amounts of fatty acids when compared to a control seed or plant, such as a seed or plant comprising a comparable polynucleotide which lacks the modification.

In certain embodiments, the linoleic acid content in the seed containing or expressing the modified MFT polypeptides disclosed herein comprises an increase of at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45% or 50% relative to the linoleic acid content of a control seed (e.g., seed expressing the polypeptide without the modifications). In certain embodiments, the linoleic acid content in the seed containing or expressing the modified polynucleotides or polypeptides disclosed herein comprises at least about a 0.1, 0.5, 1, 1.5, 2, 2.5, 3, 3.5, 4, 4.5, or 5 and less than 15, 14, 13, 12, 11, 10, 9, 8, 7, 6, 5, 4.5, 4, 3.5, 3, 2.5, 2, 1.5, 1, or 0.5 percentage point increase in linoleic acid content as compared to a control seed.

In certain embodiments, the linolenic acid content in the seed containing or expressing the modified polynucleotides or polypeptides disclosed herein comprises an decrease of at least 0.5%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45% or 50% relative to the linolenic acid content of a control seed (e.g., seed expressing the polypeptide without the modifications). In certain embodiments, the linolenic acid content in the seed containing or expressing the modified polynucleotides or polypeptides disclosed herein comprises at least about a −4, −3.5, −3, −2.5, −2, −1.5, −1, −0.5, 0%, 0.5, 1, 1.5, 2, 2.5, 3, 3.5, 4, 4.5, or 5 and less than 15, 14, 13, 12, 11, 10, 9, 8, 7, 6, 5, 4.5, 4, 3.5, 3, 2.5, 2, 1.5, 1, or 0.5 percentage point change in linolenic acid content as compared to a control seed.

In certain embodiments, the plants comprising the modified polynucleotide encoding the MFT polypeptide have a yield that is greater than or within 0.5%, 1%, 1.5%, 2%, 2.5%, 3%, 3.5%, 4%, 4.5%, or 5%, as compared to the corresponding control plant, for example, one which has a similar genetic background but lacks the introduced mutations.

As used herein, “yield” refers to the amount of agricultural production harvested per unit of land and may include reference to bushels per acre or kilograms per hectare of a crop at harvest, as adjusted for grain moisture. Grain moisture is measured in the grain at harvest. The adjusted test weight of grain is determined to be the weight in pounds per bushel or kilogram, adjusted for grain moisture level at harvest.

In certain embodiments, the soybean plants comprising the modified MFT gene coding sequence are elite soybean plant lines. In certain embodiments, the plant cells, plant parts, seeds, and grain are isolated from or produced by an elite plant line.

In certain embodiments, the modified MFT polynucleotide is operably linked to a heterologous regulatory element, such as but not limited to a constitutive, tissue-preferred, or other promoter for expression in plants or a constitutive enhancer.

In certain embodiments, the modified MFT polynucleotide described herein is introduced into the plants, plant cells, plant parts, seeds, and grain by a genetic modification at a genomic locus that encodes an endogenous MFT polypeptide, such that the plant, plant cell, plant part, seed, or grain encodes any of the modified MFT polypeptides described herein, for example, a MFT polypeptide comprising an amino acid sequence that is at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to any one of SEQ ID NOs: 2 and comprising a non-threonine at the amino acid residue corresponding to position T82 of SEQ ID NO: 2. In certain embodiments, the genomic locus that encodes an endogenous MFT polypeptide comprises a polynucleotide sequence that is at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 7. The genetic modification of the genomic locus may be done using any genome modification technique known in the art or described herein. In certain embodiments the genetic modification may be facilitated through base editing deaminases or the induction of a double-stranded break (DSB) or single-strand break, in a defined position in the genome near the desired alteration. DSBs can be induced using any DSB-inducing agent available, including, but not limited to, TALENs, meganucleases, zinc finger nucleases, Cas9-gRNA systems (based on bacterial CRISPR-Cas systems), guided cpf1 endonuclease systems, and the like.

A “genomic locus” as used herein, generally refers to the location on a chromosome of the plant where a gene, such as a polynucleotide encoding a MFT polypeptide, is found. As used herein, “gene” includes a nucleic acid fragment that expresses a functional molecule such as, but not limited to, a specific protein coding sequence and regulatory elements, such as those preceding (5′ non-coding sequences) and following (3′ non-coding sequences) the coding sequence.

In certain embodiments, the soybean plants, plant cells, plant parts, seeds, and/or grain disclosed herein can further comprise one or more traits of interest. In certain embodiments, the soybean plant, plant cell, plant part, seeds, and/or grain is stacked with any combination of polynucleotide sequences of interest in order to create plants with a desired combination of traits. As used herein, the term “stacked” refers to having multiple traits present in the same plant or organism of interest. For example, “stacked traits” may comprise a molecular stack where the sequences are physically adjacent to each other. A trait, as used herein, refers to the phenotype derived from a particular sequence or groups of sequences. In one embodiment, the molecular stack comprises at least one polynucleotide that confers tolerance to glyphosate. Polynucleotides that confer glyphosate tolerance are known in the art.

In certain embodiments, the molecular stack comprises at least one polynucleotide that confers tolerance to glyphosate and at least one additional polynucleotide that confers tolerance to a second herbicide.

In certain embodiments, the plant, plant cell, seed, and/or grain having an inventive polynucleotide sequence may be stacked with, for example, one or more sequences that confer tolerance to: an ALS inhibitor; an HPPD inhibitor; 2,4-D; other phenoxy auxin herbicides; aryloxyphenoxypropionate herbicides; dicamba; glufosinate herbicides; herbicides which target the protox enzyme (also referred to as “protox inhibitors”)

The plant, plant cell, plant part, seed, and/or grain comprising a polynucleotide sequence disclosed herein can also be combined with at least one other trait to produce plants that further comprise a variety of desired trait combinations. For instance, the plant, plant cell, plant part, seed, and/or grain having the polynucleotide sequence may be stacked with polynucleotides encoding polypeptides having pesticidal and/or insecticidal activity, or a plant, plant cell, plant part, seed, and/or grain comprising a polynucleotide sequence provided herein may be combined with a plant disease resistance gene.

In certain embodiments, the molecular stack comprises at least one additional polynucleotide that confers increased seed protein or oil content. For instance, a modified polynucleotide encoding a diacylglycerol acyltransferase (DGAT) polypeptide, such as those described in WO19/232182, or a high oleic acid trait, such as those described in U.S. Pat. No. 8,609,935.

These stacked combinations can be created by any method including, but not limited to, breeding plants by any conventional methodology, or genetic transformation. If the sequences are stacked by genetically transforming the plants, the polynucleotide sequences of interest can be combined at any time and in any order. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. In certain cases, it may be desirable to introduce a transformation cassette that will suppress the expression of the polynucleotide of interest. This may be combined with any combination of other suppression cassettes or overexpression cassettes to generate the desired combination of traits in the plant. It is further recognized that polynucleotide sequences can be stacked at a desired genomic location using a site-specific recombination system. See, for example, WO99/25821, WO99/25854, WO99/25840, WO99/25855, and WO99/25853, all of which are herein incorporated by reference.

Any plant produced or disclosed herein having a modified MFT gene sequence resulting in high oil can be used to make a food or a feed product. Such methods comprise obtaining a plant, explant, seed, plant cell, or cell comprising the modified MFT gene sequence and processing the plant, explant, seed, plant cell, or cell to produce a food or feed product.

Also provided are methods for increasing seed oil and/or protein content comprising expressing in a plant a modified MFT polynucleotide encoding a modified MFT polypeptide described herein (e.g., an MFT polypeptide comprising an amino acid sequence that is at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 2 and comprising a non-threonine at the amino acid residue corresponding to position T82 of SEQ ID NO: 2). In certain embodiments, the method comprises: expressing in a regenerable plant cell a recombinant DNA construct comprising a polynucleotide described herein; and generating the plant from the plant cell. In certain embodiments, the polynucleotide is operably linked to at least one regulatory sequence. In certain embodiments, the at least one regulatory sequence is a heterologous promoter. The recombinant DNA construct for use in the method may be any recombinant DNA construct provided herein. In certain embodiments the recombinant DNA is expressed by introducing into a plant, plant cell, plant part, seed, and/or grain the recombinant DNA construct, whereby the polypeptide is expressed in the plant, plant cell, plant part, seed, and/or grain. In certain embodiments the recombinant DNA construct is incorporated into the genome of the plant.

Various methods can be used to introduce the MFT sequences (e.g., modified MFT sequence or recombinant DNA comprising the modified MFT sequence) into a plant, plant part, plant cell, seed, and/or grain. “Introducing” is intended to mean presenting to the plant, plant cell, seed, and/or grain the inventive polynucleotide or resulting polypeptide in such a manner that the sequence gains access to the interior of a cell of the plant. The methods of the disclosure do not depend on a particular method for introducing a sequence into a plant, plant cell, seed, and/or grain, only that the polynucleotide or polypeptide gains access to the interior of at least one cell of the plant. One of skill will recognize that after the expression cassette containing the inventive polynucleotide is stably incorporated in transgenic plants and confirmed to be operable, it can be introduced into other plants by sexual crossing. Any of a number of standard breeding techniques can be used, depending upon the species to be crossed.

Also provided are methods for increasing seed oil and/or protein content comprising introducing into an endogenous MFT gene a genetic modification producing a modified MFT gene coding sequence encoding a modified MFT polypeptide described herein (e.g., an MFT polypeptide comprising an amino acid sequence that is at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 2 and comprising a non-threonine at the amino acid residue corresponding to position T82 of SEQ ID NO: 2).

In certain embodiments, the method comprises providing a guide RNA, at least one polynucleotide modification template, and at least one Cas endonuclease to a plant cell, wherein the at least one Cas endonuclease introduces a double stranded break at an endogenous MFT gene in the plant cell and generates any of the modified polynucleotides described herein, obtaining a plant from the plant cell; and generating a progeny plant that comprises the polynucleotide and produces seeds having an increased oil content as compared to a control plant not comprising the polynucleotide.

Various methods can be used to introduce the genetic modification at a genomic locus that encodes an MFT polypeptide into the plant, plant part, plant cell, seed, and/or grain. In certain embodiments the genetic modification is through a genome modification technique selected from the group consisting of a polynucleotide-guided endonuclease, CRISPR-Cas endonucleases, base editing deaminases, zinc finger nuclease, a transcription activator-like effector nuclease (TALEN), engineered site-specific meganuclease, or Argonaute.

In certain embodiments, the genetic modification may be facilitated through the induction of a double-stranded break (DSB) or single-strand break, in a defined position in the genome near the desired alteration. DSBs can be induced using any DSB-inducing agent available, including, but not limited to, TALENs, meganucleases, zinc finger nucleases, Cas9-gRNA systems (based on bacterial CRISPR-Cas systems), guided cpfl endonuclease systems, and the like. In some embodiments, the introduction of a DSB can be combined with the introduction of a polynucleotide modification template.

The process for editing a genomic sequence combining DSB and modification templates generally comprises providing to a host cell, a DSB-inducing agent, or a nucleic acid encoding a DSB-inducing agent, that recognizes a target sequence in the chromosomal sequence and is able to induce a DSB in the genomic sequence, and at least one polynucleotide modification template comprising at least one nucleotide alteration when compared to the nucleotide sequence to be edited. The polynucleotide modification template can further comprise nucleotide sequences flanking the at least one nucleotide alteration, in which the flanking sequences are substantially homologous to the chromosomal region flanking the DSB.

The endonuclease can be provided to a cell by any method known in the art, for example, but not limited to, transient introduction methods, transfection, microinjection, and/or topical application or indirectly via recombination constructs. The endonuclease can be provided as a protein or as a guided polynucleotide complex directly to a cell or indirectly via recombination constructs. The endonuclease can be introduced into a cell transiently or can be incorporated into the genome of the host cell using any method known in the art. In the case of a CRISPR-Cas system, uptake of the endonuclease and/or the guided polynucleotide into the cell can be facilitated with a Cell Penetrating Peptide (CPP) as described in WO2016073433 published May 12, 2016.

TAL effector nucleases (TALEN) are a class of sequence-specific nucleases that can be used to make double-strand breaks at specific target sequences in the genome of a plant or other organism (Miller et al. (2011) Nature Biotechnology 29:143-148).

Endonucleases are enzymes that cleave the phosphodiester bond within a polynucleotide chain. Endonucleases include restriction endonucleases, which cleave DNA at specific sites without damaging the bases, and meganucleases, also known as homing endonucleases (HEases), which like restriction endonucleases, bind and cut at a specific recognition site, however the recognition sites for meganucleases are typically longer, about 18 bp or more (WO2012129373). Meganucleases have been classified into four families based on conserved sequence motifs. These motifs participate in the coordination of metal ions and hydrolysis of phosphodiester bonds. HEases are notable for their long recognition sites, and for tolerating some sequence polymorphisms in their DNA substrates. The naming convention for meganuclease is similar to the convention for other restriction endonuclease. Meganucleases are also characterized by prefix F—, I—, or PI— for enzymes encoded by free-standing ORFs, introns, and inteins, respectively. One step in the recombination process involves polynucleotide cleavage at or near the recognition site. The cleaving activity can be used to produce a double-strand break. In some examples the recombinase is from the Integrase or Resolvase families.

Zinc finger nucleases (ZFNs) are engineered double-strand break inducing agents comprised of a zinc finger DNA binding domain and a double-strand-break-inducing agent domain. Recognition site specificity is conferred by the zinc finger domain, which typically comprising two, three, or four zinc fingers, for example having a C2H2 structure, however other zinc finger structures are known and have been engineered. Zinc finger domains are amenable for designing polypeptides which specifically bind a selected polynucleotide recognition sequence. ZFNs include an engineered DNA-binding zinc finger domain linked to a non-specific endonuclease domain, for example nuclease domain from a Type IIs endonuclease such as FokI. Additional functionalities can be fused to the zinc-finger binding domain, including transcriptional activator domains, transcription repressor domains, and methylases. In some examples, dimerization of nuclease domain is required for cleavage activity. Each zinc finger recognizes three consecutive base pairs in the target DNA. For example, a 3-finger domain recognized a sequence of 9 contiguous nucleotides, with a dimerization requirement of the nuclease, two sets of zinc finger triplets are used to bind an 18-nucleotide recognition sequence.

Genome editing using DSB-inducing agents, such as Cas9-gRNA complexes, has been described, for example in U.S. Patent Application US 2015-0082478 A1, WO2015/026886 A1, WO2016007347, and WO201625131 all of which are incorporated by reference herein.

In certain embodiments the genetic modification is introduced without introducing a double strand break using base editing technology.

In certain embodiments, base editing comprises (i) a catalytically impaired CRISPR-Cas9 mutant that is mutated such that one of their nuclease domains cannot make DSBs; (ii) a single-strand-specific cytidine/adenine deaminase that converts C to U or A to G within an appropriate nucleotide window in the single-stranded DNA bubble created by Cas9; (iii) a uracil glycosylase inhibitor (UGI) that impedes uracil excision and downstream processes that decrease base editing efficiency and product purity; or (iv) nickase activity to cleave the non-edited DNA strand, followed by cellular DNA repair processes to replace the G-containing DNA strand.

Further provided is a method for producing, generating, and/or identifying high oil MFT mutant seeds comprising detecting in a mutant seed library the presence of one or more MFT mutant seeds, the one or more MFT mutant seeds comprising a modified MFT gene having at least 95% identity to SEQ ID NO: 7, assaying the seed oil content of the one or more MFT mutant seeds, selecting from the one or more MFT mutant seeds at least one MFT mutant seed having increased seed oil content as compared to a control seed not comprising the modified MFT gene, and crossing a plant grown from the selected MFT mutant seed with a second soybean plant to produce a progeny population, the progeny population comprising at least one plant having increased seed oil content as compared to a seed of a control plant and comprising the modified MFT gene. In certain embodiments, the method further comprises genotyping the progeny population for the presence of at least one marker genetically linked to a locus comprising or corresponding to the modified MFT gene, the at least one marker detecting a modification in the MFT gene, and selecting from the progeny population one or more soybean plants comprising the at least one marker. In certain embodiments, the second soybean plant is an elite soybean variety.

In certain embodiments, the method further comprises generating the mutant seed library for use in the methods described herein by treating a population of seed with a mutagen to produce a mutant population of seeds. As used herein, a “mutagen” refers to any agent that causes a genetic mutation in the genetic material of the treated seed and plant grown therefrom. In certain embodiments, the mutagen is radiation or a chemical mutagen.

In certain embodiments, the mutagen is a chemical mutagen. The type of chemical mutagen is not particularly limited and can be selected by a person of ordinary skill in the art based upon the number and types of mutations desired. In certain embodiments, the chemical mutagen is one or more of base analogues, 5-bromo-uracil, 8-ethoxy caffeine, antibiotics, alkylating agents, sulfur mustards, nitrogen mustards, epoxides, ethylenamines, sulfates, sulfonates, sulfones, lactones, azide, hydroxylamine, nitrous acid, and acridines.

In certain embodiments, the mutagen is radiation. The type of radiation is not particularly limited and can be selected by a person of ordinary skill in the art based upon the number and types of mutations desired. In certain embodiments, the radiation is one or more of x-rays, gamma rays, neutrons, beta radiation, and ultraviolet radiation. In certain embodiments, the mutagen is a gamma ray. In certain embodiments, the gamma ray is administered to the seed at dose of at least 50 gray (Gy), 60 Gy, 70 Gy, 80 Gy, 90 Gy, 100 Gy, 120 Gy, 140 Gy, 160 Gy, 180 Gy, 200 Gy, 225 Gy, 250 Gy, 275 Gy, 300 Gy, 325 Gy, 350 Gy, 375 Gy, 400 Gy, 450 Gy, 500 Gy, 550 Gy, 600 Gy, 650 Gy, or 700 Gy) and less than 1500 Gy, 1400 Gy, 1300 Gy, 1200 Gy, 1100 Gy, 1000 Gy, 950 Gy, 900 Gy, 850 Gy, 800 Gy, 750 Gy, 700 Gy, 650 Gy, 600 Gy, 550 Gy, 500 Gy, 450 Gy, 400 Gy, 350 Gy, 300 Gy, 250 Gy, or 200 Gy. The gray (Gy) is a derived unit of ionizing radiation dose in the International System of Units (SI) as the absorption of one joule of radiation energy per kilogram of matter.

The seed oil content of the one or more MFT mutant seeds can be measured (assayed) using any method known in the art. In certain embodiments, the seed oil content is measured using a non-destructive chemical analysis such as, for example, a near infrared spectroscopy (NIRS) method such as near infrared reflectance (NIR), near infrared transmittance (NIT), single seed NIR (SS—NIR), bulk NIT, or Fourier transform NIR (FT-NIR).

In certain embodiments, the plant generated from the methods described herein produces seeds having an increase in total oil content when compared to a seed or plant comprising a comparable polynucleotide which lacks the modification.

In certain embodiments, the oil content in the seeds of the plants produced by the methods described herein comprise an increase of at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45% or 50% relative to the oil content measured on a dry weight basis, or adjusted to 13% moisture, of a control seed (e.g., seed expressing the polypeptide without the modifications). In certain embodiments, the oil content in the seeds of the plants produced by the methods described herein comprise at least about a 0.1, 1.5, 2, 2.5, 3, 3.5, 4, 5, 10, or 15 and less than 20, 15, 10, 9, 8, 7, 6, or 5 percentage point increase in total oil measured on a dry weight basis, or adjusted to 13% moisture, as compared to a control seed (e.g., seed comprising a non-modified polypeptide).

In certain embodiments, the plant generated from the methods described herein produce seeds having an increase in total protein content when compared to a seed or plant comprising a comparable polynucleotide which lacks the modification.

In certain embodiments, the protein content in the seeds of the plants produced by the methods described herein comprise an increase of at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45% or 50% relative to the protein content measured on a dry weight basis, or adjusted to 13% moisture, of a control seed (e.g., seed expressing the polypeptide without the modifications). In certain embodiments, the protein content in the seeds of the plants produced by the methods described herein comprise at least about a 0.1, 0.5, 1, 1.5, 2, 2.5, 3, 3.5, 4, 4.5, or 5 and less than 15, 14, 13, 12, 11, 10, 9, 8, 7, 6, 5, 4.5, 4, 3.5, 3, 2.5, 2, 1.5, 1, or 0.5 percentage point change in total protein measured on a dry weight basis, or adjusted to 13% moisture, as compared to a control seed (e.g., seed comprising a non-modified polypeptide).

In certain embodiments, the plants generated from the methods described herein produce seeds having an increase in both total protein and total oil content when compared to a seed or plant comprising a comparable polynucleotide which lacks the modification. The increase in total oil content and total protein content can be any increase described herein.

In certain embodiments, the plants generated from the methods described have a yield that is greater than or within 0.5%, 1%, 1.5%, 2%, 2.5%, 3%, 3.5%, 4%, 4.5%, or 5%, as compared to the corresponding control plant, for example, one which has a similar genetic background but lacks the introduced mutations.

In certain embodiments, the method further comprises growing seed comprising the introduced genetic modification to produce a second-generation progeny plant that comprises the modified MFT polypeptide and backcrossing the second-generation progeny plant to the second plant to produce a backcross progeny plant that comprises the modified MFT polypeptide and produces backcrossed seed with increased oil content. The increase in seed oil and/or protein may be any increase described herein. In certain embodiments, the seed has a modified amount of fatty acids as described herein. In certain embodiments, the plants have a yield that is greater than or within 0.5%, 1%, 1.5%, 2%, 2.5%, 3%, 3.5%, 4%, 4.5%, or 5%, as compared to the corresponding control plant.

The present disclosure is further illustrated in the following embodiments. These embodiments are given by way of illustration only.

Embodiment 1: A method for producing a soybean plant having high seed oil, the method comprising: (a) genotyping a soybean population comprising a plurality of soybean plants or soybean germplasm for the presence of at least one marker genetically linked to a locus comprising or corresponding to an MFT gene having at least 95% identity to SEQ ID NO: 7, the at least one marker detecting a modification in the MFT gene; (b) selecting from the soybean population one or more soybean plants or soybean germplasm comprising the at least one marker; and (c) crossing the selected soybean plant or soybean germplasm with a second soybean plant or soybean germplasm to produce a progeny population, wherein at least one soybean plant or soybean germplasm of the progeny population comprises the at least one marker and has high seed oil as compared to a seed of a control plant.

Embodiment 2: The method of embodiment 1, wherein the at least one marker comprises an insertion, deletion, polymorphism, or any combination thereof in a coding sequence of the MFT gene.

Embodiment 3: The method of embodiment 1 or 2, wherein the at least one marker comprises an insertion, deletion, polymorphism, or any combination thereof in a regulatory sequence of the MFT gene.

Embodiment 4: The method of embodiment 3, wherein the MFT gene regulatory sequence comprises an MFT promoter sequence having at least 95% sequence identity to positions 1-1431 of SEQ ID NO: 7.

Embodiment 5: The method of any one of embodiments 1-4, wherein the at least one marker comprises a polymorphism corresponding to a CC at marker S101AY8-00-Q002, a T at marker S2000A7-001-Q001, a 4 base pair deletion at marker S2000A3-001-Q001, a 1 base pair deletion at marker S2000A4-001-Q001, and a G insertion at marker S2000A5-001-Q001.

Embodiment 6: The method of any one of embodiments 1-5, further comprising detecting a second marker genetically linked to the locus comprising or corresponding to the MFT gene.

Embodiment 7: The method of any one of embodiments 1-6, wherein genotyping comprises amplifying a nucleic acid sequence comprising the at least one marker and detecting the resulting amplified nucleic acid comprising the marker.

Embodiment 8: The method of embodiment 7, wherein the amplified nucleic acid comprises at least a portion of a sequence corresponding to SEQ ID NO: 8, 18, 28, 34, or 40.

Embodiment 9: The method of embodiment 7 or 8, wherein the amplifying the nucleic acid sequence comprises providing nucleic acid primers, wherein the nucleic acid primers comprise one or more of SEQ ID NOs: 10, 11, 20, 21, 30, 31, 36, 37, 42, and 43.

Embodiment 10: The method of any one of embodiments 7-9, wherein detecting the resulting amplified nucleic acid further comprises hybridizing the resulting amplified nucleic acid with one or more nucleic acid probes, wherein the nucleic acid probes comprise one or more of SEQ ID NOs: 9, 19, 29, 35 and 41.

Embodiment 11: A method for producing a population of soybean plants or soybean germplasm having an increased seed oil content, the method comprising: (a) crossing a first soybean plant or first soybean germplasm comprising a modification at a locus comprising or corresponding to an MFT gene having at least 95% identity to SEQ ID NO: 7, the modification decreasing the expression or activity of an encoded MFT polypeptide, with a second soybean plant or second soybean germplasm to form a soybean plant or soybean germplasm population; (b) genotyping the soybean plant or soybean germplasm population for the presence of at least one marker genetically linked to the locus, the at least one marker detecting the modification; and (c) selecting from the soybean population one or more soybean plants or soybean germplasm comprising the at least one marker.

Embodiment 12: The method of embodiment 11, wherein the at least one marker comprises an insertion, deletion, polymorphism, or any combination thereof in a coding sequence of the MFT gene.

Embodiment 13: The method of embodiment 11 or 12, wherein the at least one marker comprises an insertion, deletion, polymorphism, or any combination thereof in a regulatory sequence of the MFT gene.

Embodiment 14: The method of embodiment 13, wherein the MFT gene regulatory sequence comprises an MFT promoter sequence having at least 95% sequence identity to positions 1-1431 of SEQ ID NO: 7.

Embodiment 15: The method of any one of embodiments 11-14, wherein the at least one marker comprises a polymorphism corresponding to a CC at marker S101AY8-00-Q002, a T at marker S2000A7-001-Q001, a 4 base pair deletion at marker S2000A3-001-Q001, a 1 base pair deletion at marker S2000A4-001-Q001, and a G insertion at marker S2000A5-001-Q001.

Embodiment 16: The method of any one of embodiments 11-15, further comprising detecting a second marker genetically linked to the locus comprising or corresponding to the MFT gene.

Embodiment 17: The method of any one of embodiments 11-16, wherein genotyping comprises amplifying a nucleic acid sequence comprising the at least one marker and detecting the resulting amplified nucleic acid comprising the marker.

Embodiment 18: The method of embodiment 17, wherein the amplified nucleic acid comprises at least a portion of a sequence corresponding to SEQ ID NO: 8, 18, 28, 34, or 40.

Embodiment 19: The method of embodiment 17 or 18, wherein the amplifying the nucleic acid sequence comprises providing nucleic acid primers, wherein the nucleic acid primers comprise one or more of SEQ ID NOs: 10, 11, 20, 21, 30, 31, 36, 37, 42, and 43.

Embodiment 20: The method of any one of embodiments 17-19, wherein detecting the resulting amplified nucleic acid further comprises hybridizing the resulting amplified nucleic acid with one or more nucleic acid probes, wherein the nucleic acid probes comprise one or more of SEQ ID NOs: 9, 19, 29, 35 and 41.

Embodiment 21: A method of introgressing a high soybean seed oil MFT allele into a soybean plant, the method comprising: (a) crossing a first soybean plant with a second soybean plant to produce a progeny population, the first soybean plant comprising a high soybean seed oil MFT allele comprising a modification of an MFT gene comprising a polynucleotide sequence having at least 95% sequence identity with SEQ ID NO: 7, the modification decreasing the expression or activity of an MFT polypeptide encoded by the MFT gene; (b) genotyping the progeny population for the presence of at least one marker genetically linked to the high soybean seed oil MFT allele; and (c) selecting progeny that comprise the at least one marker to obtain soybean plants comprising the high soybean seed oil MFT allele.

Embodiment 22: The method of embodiment 21, wherein the modification is polymorphism that decreases expression of a polypeptide encoded by the MFT gene compared to a wild-type polypeptide.

Embodiment 23: The method of embodiment 21, wherein the modification is a polymorphism that decreases activity of a polypeptide encoded by the MFT gene, compared to a wild-type polypeptide.

Embodiment 24: The method of any one of embodiments 21-23 wherein a soybean seed of a soybean plant selected from the progeny population has an oil content that is increased by at least a 1 percentage point, a protein content that is increased by at least a 0.25 percentage point, or a combination thereof, as compared to a control soybean seed when measured at 13% moisture content.

Embodiment 25: The method of any one of embodiments 21-24, wherein the at least one marker genetically linked to the high oil MFT allele is within 20 centimorgans of the high oil MFT allele.

Embodiment 26: The method of any one of embodiments 21-25, wherein the at least one marker comprises a polymorphism corresponding to a CC at marker S101AY8-00-Q002, a T at marker S2000A7-001-Q001, a 4 base pair deletion at marker S2000A3-001-Q001, a 1 base pair deletion at marker S2000A4-001-Q001, a G insertion at marker S2000A5-001-Q001, a T at position 38012490 on Chr05, an A at position 39924818 on Chr05, a T at position 40892689 on Chr05, a C at position 41265253 on Chr05, a G at position 41673315 on Chr05, and a C at position 42136562 on Chr05.

Embodiment 27: A soybean cell having an increased oil content and comprising a modified MFT gene coding sequence encoding a modified MFT polypeptide comprising an amino acid sequence that is at least 95% identical to SEQ ID NO: 2 and comprises a non-threonine at the amino acid residue corresponding to position 82 of SEQ ID NO: 2.

Embodiment 28: The soybean cell of embodiment 27, wherein the modified MFT polypeptide sequence comprises a glycine, asparagine, glutamine, alanine, serine, or cysteine at the amino acid residue corresponding to position 82 of SEQ ID NO: 2.

Embodiment 29: The soybean cell of embodiment 27 or 28, wherein the modified MFT polypeptide further comprises a non-leucine at the amino acid residue corresponding to position L140 of SEQ ID NO: 2.

Embodiment 30: The soybean cell of embodiment 29, wherein the modified MFT polypeptide sequence comprises a glycine, asparagine, glutamine, alanine, serine, cysteine, or threonine at the amino acid residue corresponding to position L140 of SEQ ID NO: 2.

Embodiment 31: The soybean cell of embodiment 29 or 30, wherein the modified MFT polypeptide sequence comprises a glycine, asparagine, glutamine, alanine, serine, cysteine, or threonine at the amino acid residue corresponding to position L140 of SEQ ID NO: 2 and a glycine, asparagine, glutamine, alanine, serine, or cysteine at the amino acid residue corresponding to position 82 of SEQ ID NO: 2.

Embodiment 32: A soybean plant comprising the soybean cell of any one of embodiments 27-31.

Embodiment 33: A soybean seed comprising the soybean cell of any one of embodiments 27-31.

Embodiment 34: The soybean seed of embodiment 33, wherein the oil content of the soybean seed is increased by at least a 1 percentage point, the protein content of the soybean seed is increased by at least a 0.25 percentage point, or a combination thereof, as compared to a control soybean seed when measured at 13% moisture content.

Embodiment 35: A soybean plant comprising soybean seeds having increased oil content as compared with control seeds of a control plant when measured at 13% seed moisture content, the soybean plant comprising a modified MFT gene coding sequence encoding a modified MFT polypeptide comprising an amino acid sequence that is at least 95% identical to SEQ ID NO: 2 and comprises a non-threonine at the amino acid residue corresponding to position 82 of SEQ ID NO: 2 or a combination thereof.

Embodiment 36: The soybean plant of claim 35, wherein the modified MFT polypeptide sequence comprises a glycine, asparagine, glutamine, alanine, serine, or cysteine at the amino acid residue corresponding to position 82 of SEQ ID NO: 2.

Embodiment 37: The soybean plant of claim 35 or 36, wherein the soybean plant further comprises a non-leucine at the amino acid residue corresponding to position L140 of SEQ ID NO: 2.

Embodiment 38: The soybean plant of claim 37, wherein the modified MFT polypeptide sequence comprises a glycine, asparagine, glutamine, alanine, serine, cysteine, or threonine at the amino acid residue corresponding to position L140 of SEQ ID NO: 2.

Embodiment 39: The soybean plant of claim 37 or 38, wherein the modified MFT polypeptide sequence comprises a glycine, asparagine, glutamine, alanine, serine, cysteine, or threonine at the amino acid residue corresponding to position L140 of SEQ ID NO: 2 and a glycine, asparagine, glutamine, alanine, serine, or cysteine at the amino acid residue corresponding to position 82 of SEQ ID NO: 2.

Embodiment 40: The soybean plant of any one of claims 35-39, wherein the soybean seeds further comprise at least at least a 1 percentage point increase in oil content, a 0.25 percentage point increase in protein content, or a combination thereof, as compared to the control seeds when measured at 13% moisture content.

Embodiment 41: A method of producing the soybean plant of any one of claims 35-40, the method comprising introducing into an endogenous MFT gene a modification producing the modified MFT gene coding sequence encoding the modified MFT polypeptide.

Embodiment 42: A method for identifying a high seed oil MFT mutant sequence, the method comprising: (a) detecting in a sequenced high seed oil mutant library the presence of one or more modified MFT sequences corresponding to an MFT gene having at least 95% identity to SEQ ID NO: 7; (b) expressing the one or more modified MFT sequences from the sequenced high seed oil mutant library in a plant; and (c) assaying a seed of the plant expressing the one or more modified MFT sequences, the seed having increased oil content as compared to seed of a control plant not comprising the modified MFT sequence.

Embodiment 43: A method for identifying an MFT mutant, the method comprising: (a) detecting MFT mutant lines in a sequenced mutant library containing the presence of one or more modified MFT sequences corresponding to an MFT gene having at least 95% identity to SEQ ID NO: 7; (b) assaying for increased seed oil content in isolated MFT mutants; and (c) integrating an MFT mutant into an elite soybean variety by using an MFT gene specific molecular marker or an MFT flanking molecular marker, the elite variety having increased oil content as compared to seed of a control plant not comprising the modified MFT sequence.

Embodiment 44: A method for producing high oil MFT mutant seeds, the method comprising: (a) detecting in a mutant seed library the presence of one or more MFT mutant seeds, the one or more MFT mutant seeds comprising a modified MFT gene having at least 95% identity to SEQ ID NO: 7; (b) assaying the seed oil content of the one or more MFT mutant seeds; (c) selecting from the one or more MFT mutant seeds at least one MFT mutant seed having increased seed oil content as compared to a control seed not comprising the modified MFT gene; and (d) crossing a plant grown from the selected MFT mutant seed with a second soybean plant to produce a progeny population, the progeny population comprising at least one plant having increased seed oil content as compared to a seed of a control plant and comprising the modified MFT gene.

Embodiment 45: The method of embodiment 44, wherein the second soybean plant is an elite soybean variety.

Embodiment 46: The method of embodiment 44 or 45, wherein the method further comprises genotyping the progeny population for the presence of at least one marker genetically linked to a locus comprising or corresponding to the modified MFT gene, the at least one marker detecting a modification in the MFT gene, and selecting from the progeny population one or more soybean plants comprising the at least one marker.

The following are examples of specific embodiments of some aspects of the invention. The examples are offered for illustrative purposes only and are not intended to limit the scope of the invention in any way.

### Example 1

This example demonstrates the isolation and characterization of a modified MFT gene that increases seed oil protein content.

Using a high throughput single seed screening method, a high protein and oil mutant, from an ethyl methanesulfonate (EMS) mutagenized population was identified and is referred to as EHPT11. M₂ plants were grown out in a Puerto Rico winter nursery in 2021 and a test of the M_{2:3} EHPT11 seeds determined that the EHPT11 seeds had a higher protein and oil content when compared to the control wild type seed. M₃ plants were grown out in a Johnston field in short rows in 2022. The EHPT11 M_{3:4} seeds showed a significant increase in seed oil and protein content. Overall, the EHPT11 seeds had an increase in seed protein+oil by 2.1-3.8 points with no inverse correlation between protein and oil in 2-year field tests (Table 2).

TABLE 2
Seed oil and protein content of EHPT11 mutant

| | M3 2021 Puerto Rico field | | | M4 2022 Johnston field | | |
|---|---|---|---|---|---|---|
| | WT | EHPT11 | Diff | WT | EHPT11 | Diff |
| Seed oil % | 21.5 | 22.5 | 1.0 | 20.4 | 20.5 | 0.1 |
| Seed protein % | 34.0 | 36.8 | 2.8 | 33.1 | 35.1 | 2.0 |
| Protein + oil % | 55.5 | 59.3 | 3.8 | 53.5 | 55.6 | 2.1 |

Note: Seed oil and protein content is adjusted to 13% seed moisture basis.

To identify the causative mutation responsible for high protein and oil, DNA was isolated from EHPT11 mutant and was subjected to whole-genome sequencing on the Illumina platform. Raw Illumina reads were processed using custom internal scripts (SNPfinder pipeline) which performs read mapping and detection of sequence variants (specifically single nucleotide polymorphisms (SNPs) or short Insertions or deletions (InDels) (~50 bp or less). In addition to identifying SNPs and short InDels, the Illumina sequencing data were also analyzed using custom internal pipelines to identify large deletions (greater than 500 bp) in the genomic sequence of the soy mutant plants. Compared to wild type reference genome, 24 non-synonymous mutations which resulted in an amino acid change in the protein were identified (Table 3). One of the 24 candidate genes is Glyma.05g244100 encoding a Mother of FT (flowering time) and TFL1 (terminated flowering locus1) (MFT)-like protein. This gene was validated as a causative gene responsible for high protein and oil in the HiPO-538 mutant (WO2021/252283). The EHPT11 mutant contains a single amino acid mutation from threonine to serine residue at position 82. Because both the EHPT11 and HiPO-538 mutants showed a similar high oil and protein phenotype, EHPT11 most likely is an independent second allele of the HiPO-538 mutant and indicates that other MFT mutant alleles could be identified from mutant populations to increase seed oil and protein content in soybean.

TABLE 3
Non-synonymous mutations identified in EHPT 11 mutant

| Gene | Function annotation |
|---|---|
| Glyma.03G008500.1 | Uncharacterized protein (Fragment) |
| Glyma.03G074300.1 | Legume lectin domain containing protein |
| Glyma.04G130300.1 | Putative MYST-like histone acetyltransferase 1 |
| Glyma.05G244100.1 | protein MOTHER of FT and TFL1 homolog 1-like |
| Glyma.07G213900.1 | Protein kinase domain |
| Glyma.07G234800.1 | putative pentatricopeptide repeat-containing protein At1g69350, |
| | mitochondrial isoform X1 |
| Glyma.07G239200.1 | Putative enoyl-CoA hydratase 1 peroxisomal isoform X1 |
| Glyma.08G132800.1 | homeobox-leucine zipper protein ATHB-6-like isoform X2 |
| Glyma.13G343300.1 | Uncharacterized protein |
| Glyma.10G032200.1 | photosystem II reaction center W protein, chloroplastic-like isoform X1 |
| Glyma.10G037300.3 | Structural maintenance of chromosomes protein |
| Glyma.11G144300.1 | 11S globulin seed storage protein 2-like |
| Glyma.11G186600.1 | uncharacterized protein LOC109002419 isoform X2 |
| Glyma.12G211900.1 | Peroxidase P7-like |
| Glyma.20G192800.7 | monocopper oxidase-like protein SKU5 isoform X2 |
| Glyma.15G191500.1 | FAS1 domain-containing protein SELMODRAFT_448915-like |
| Glyma.15G230900.1 | putative disease resistance RPP13-like protein 1 isoform X2 |
| Glyma.05G078700.3 | Uncharacterized protein (Fragment) |
| Glyma.17G262700.1 | pentatricopeptide repeat-containing protein At2g13600-like isoform X1 |
| Glyma.17G133600.1 | FAM10 family protein At4g22670-like isoform X1 |
| Glyma.18G282800.1 | importin beta-like SAD2 isoform X1 |
| Glyma.19G152800.3 | BSD domain-containing protein 1-A |
| Glyma.19G234400.1 | acyl-CoA-binding domain-containing protein 4 isoform X1 |
| Glyma.20G104900.5 | Zinc finger, RING-type domain containing protein |

These data demonstrate that the EHPT11 mutant line has increased protein and oil content as compared to a control line

### Example 2

This example demonstrates the identification and characterization of markers to identify a high oil MFT mutant gene encoding an MFT polypeptide containing the leucine to serine mutation at position 140 (L140S).

A unique genotyping assay was developed to selectively detect a variant of an MFT gene containing a 2 bp mutation that encodes a polypeptide comprising a serine at the amino acid residue corresponding to position 140 of SEQ ID NO: 2 and is associated with high seed oil content. The genotyping assay combines two separate assays-S101AY8-00-Q002. The first assay M (mutant-S101AY8-00-Q002 high oil from Table 1B and Table 4) detects the mutation (VIC) while the W (wildtype-S101AY8-00-Q002 wild-type from Table 1B and Table 4) assay (FAM), detects the wild type. Together these two assays in one well of a genotyping PCR reaction (Such as TaqMan assay described here) were used as a co-dominant marker to discriminate the high protein and low protein alleles in all zygocity states. This assay is effective for foreground selection in the marker assisted back cross breeding as well as in trait purity applications.

### Example 3

This example demonstrates the identification and characterization of markers to identify a high oil MFT mutant gene encoding an MFT polypeptide containing a threonine to serine substitution at position 82 (T82S).

To selectively detect a variant of an MFT gene containing a SNP that encodes a polypeptide comprising a serine at the amino acid residue corresponding to position 82 of SEQ ID NO: 2 a unique genotyping marker was designed-S2000A7-001-Q001 (Table 1B and Table 4). A “T” allele is associated with the T82S mutant (FAM), while an “A” allele detects wild type (VIC). This marker will be used to discriminate the high oil and low oil alleles in all zygocity states. This assay is expected to be effective for foreground selection in the marker assisted back cross breeding as well as in trait purity applications.

### Example 4

The example demonstrates the identification and characterization of markers to identify a high oil MFT mutant comprising type II CRISPR/Cas edits introduced into the MFT gene.

To selectively detect MFT gene variants comprising introduced CRISPR/Cas edits, 3 assays were designed to the indels generating frame shift mutations.

S2000A3-001-Q001 (Table 1B and Table 4) was designed to detect the frame shift variant E1.10A. A deletion or “D” genotyping call is associated with the high oil phenotype, while a lack of deletion or “I” is associated with the wild-type phenotype.

S2000A4-001-Q001 (Table 1B and Table 4) was designed to detect the frame shift variants E1.2A and E1.5A. A deletion or “D” genotyping call is associated with the high oil phenotype, while a lack of deletion or “I” is associated with the wild-type phenotype.

S2000A5-001-Q001 (Table 1B and Table 4) was designed to detect the frame shift variant E1.8A. An insertion or “I” genotyping call is associated with the high oil phenotype, while a lack of insertion or “D” is associated with the wild-type phenotype.

This assay is expected to be effective for foreground selection in the marker assisted back cross breeding as well as in trait purity applications.

TABLE 4
Genomic features of the SNP markers

| | | | | | | Probe |
|---|---|---|---|---|---|---|
| | | | | | | Orientation |
| | | | | Donor- | RP/ | (positive (+) or |
| | | Physical | Genetic | High | Wild- | negative (−) |
| Marker | Chrom | Position | Position | Oil | type | strand) |
| S101AY8-00-Q002 | Chr05 | 41853325- | 122.04 | M (CC | W (TG | − |
| (2 bp mutation in MFT gene | | 41853326 | | alleles) | alleles) | |
| encoding for L140S) | | | | | | |
| S2000A7-001-Q001 | Chr05 | 41854680 | 122.05 | T | A | − |
| (1 bp mutation in MFT gene | | | | | | |
| encoding for T82S) | | | | | | |
| S2000A3-001-Q001 | Chr05 | 41854852 | 122.05 | D | I | − |
| (4 bp CRISPR deletion at pos | | | | | | |
| 201-204 of SEQ ID NO: 26) | | | | | | |
| S2000A4-001-Q001 | Chr05 | 41854855 | 122.05 | D | I | − |
| (1 bp deletion of an A at pos | | | | | | |
| 201 of SEQ ID NO: 32) | | | | | | |
| S2000A5-001-Q001 | Chr05 | 41854856 | 122.05 | I | D | − |
| (1 bp insertion of a G at pos | | | | | | |
| 201 of SEQ ID NO: 38) | | | | | | |

### Example 5

The example demonstrates the identification and characterization of markers to identify a high oil MFT mutant.

To discover any naturally occurring variation in the MFT gene and flanking sequences, Corteva's proprietary SNP database was mined. This database contained 2457 soybean elite and public lines representing North America and Latin America. 44 SNPs with very low minor allele frequency within the glyma.05g244100 gene were selected and can be converted into genotyping assays (Table 5). Of the 44 SNPs with very low minor allele frequency, 4 report non-synonymous amino acid changes in the MFT protein. The minor allele frequencies (MAF) of the SNPs within the gene ranged from 0.09 to 2.33. An additional 6 SNP flanking markers were identified which can be converted into genotyping assays to distinguish between the high oil and wild-type alleles (Table 5). Marker assays can be developed using this information, including but not limited to any one or more of sequencing or marker methods. In one example, sample tissue, including tissue from soybean leaves or seeds can be screened with the markers using a TAQMAN® PCR assay system (Life Technologies, Grand Island, NY, USA).

The TaqMan assays will be developed as follow: Primers are designed using a software program. Probes are designed using Primer Express Software. 1.5 ul of the 1:100 DNA dilution is used in the assay mix. 18 μM of each probe, and 4 μM of each primer is combined to make each assay. 13.6 ul of the assay mix is combined with 1000 ul of 1× BHQ Master Mix (Biosearch Technologies). A Meridian (Kbio) liquid handler dispenses 1.3 ul of the mix onto a 1536 plate containing ~6 ng of dried DNA. The plate is sealed with a Phusion laser sealer and thermocycle using a Kbio Hydrocycler with the following conditions: 94 C for 15 min, 40 cycles of 94 C for 30 sec, 60 C for 1 min. The excitation at wavelengths 485 (FAM) and 520 (VIC) is measured with a Pherastar plate reader. The values are normalized against ROX and plotted and scored on scatterplots utilizing the KRAKEN software.

An association analysis will be completed using the genotypic scores from the assays and the oil phenotypes of a subset of the 2457 individuals to validate the impact of these SNP's on the oil phenotypes.

TABLE 5
Naturally occurring SNPs in the MFT gene and flanking sequences

| SEQ | | | | | High | Wild |
|---|---|---|---|---|---|---|
| ID | | | Flanking | Flanking | Oil | type |
| NO: | Chromosome | Position | Pos | Sequence | Allele | Allele |
| 44 | Chr05 | 38012490 | 20 cM | CATTAGTCACTGC | T | C |
| | | | | TAAGGAGATACT[T | | |
| | | | | /C]CTGTATTTTGCT | | |
| | | | | GACAGCGGAAGGT | | |
| 45 | Chr05 | 39924818 | 10 cM | GCCTATAATAAAA | A | G |
| | | | | GTTGAGATGATT[T | | |
| | | | | /C]GAGTTTATTACT | | |
| | | | | TTATTTGCCATTT | | |
| 46 | Chr05 | 40892689 | 5 cM | CGTCCCTTGTTAG | T | A |
| | | | | GAGTTAATAACA[T | | |
| | | | | /A]AAGAATTGGGG | | |
| | | | | TCAATTCGCAACT | | |
| | | | | C | | |
| 47 | Chr05 | 41265253 | 3 cM | AAAATTATACTAG | C | T |
| | | | | TCCCCTTGATTT[C/ | | |
| | | | | T]TCCACCCGTAAA | | |
| | | | | ACTATCAGTATGG | | |
| 48 | Chr05 | 41673315 | 1 cM | GAAACTGAAGGCA | G | C |
| | | | | GTGAAAGGCGCA[ | | |
| | | | | G/C]TCAAAGACGA | | |
| | | | | ACTCAGATCAACA | | |
| | | | | GA | | |
| 49 | Chr05 | 41852875 | 0 cM | GTACCCACAACAG | G | C |
| | | | | TAAGGATAAATT[ | | |
| | | | | G/C]GAAGGATAAA | | |
| | | | | CACATACATATAC | | |
| | | | | AC | | |
| 50 | Chr05 | 41853015 | 0 cM | ATCAAAGCCGGGG | C | T |
| | | | | AAATGGTCGGCA[ | | |
| | | | | C/T]CACTCATGTCT | | |
| | | | | TGGACAACTATAT | | |
| | | | | T | | |
| 51 | Chr05 | 41853022 | 0 cM | CCGGGGAAATGGT | T | G |
| | | | | CGGCACCACTCA[T | | |
| | | | | /G]GTCTTGGACAA | | |
| | | | | CTATATTACTTTAC | | |
| 52 | Chr05 | 41853074 | 0 cM | CACACCCTCTCCC | G | T |
| | | | | TGCATTATTTAC[G/ | | |
| | | | | T]CACTAGTCATAG | | |
| | | | | ATTTTATATAGTT | | |
| 53 | Chr05 | 41853114 | 0 cM | TTTATATAGTTCTG | G | T |
| | | | | ATATCTTAAAA[G/ | | |
| | | | | T]TATATTCCAACC | | |
| | | | | ATGCATATCTCAA | | |
| 54 | Chr05 | 41853142 | 0 cM | TATTCCAACCATG | G | A |
| | | | | CATATCTCAAGA[G | | |
| | | | | /A]AGACACTGATG | | |
| | | | | GTTACAATATAGC | | |
| | | | | T | | |
| 55 | Chr05 | 41853155 | 0 cM | CATATCTCAAGAG | T | A |
| | | | | AGACACTGATGG[ | | |
| | | | | T/A]TACAATATAG | | |
| | | | | CTAGATTCAGCGC | | |
| | | | | CT | | |
| 56 | Chr05 | 41853190 | 0 cM | GCTAGATTCAGCG | A | G |
| | | | | CCTCTTAACAGC[A | | |
| | | | | /G]GGTTCTTTTTGA | | |
| | | | | GAGTTGAAGTAGA | | |
| 57 | Chr05 | 41853265 | 0 cM | CCAATTGCCTGGC | G | A |
| | | | | AAAATAACGAGT[ | | |
| | | | | G/A]TTGAAGCTTG | | |
| | | | | CTCGAGTTGGTGG | | |
| | | | | TT | | |
| 58 | Chr05 | 41853277 | 0 cM | CAAAATAACGAGT | T | A |
| | | | | GTTGAAGCTTGC[T | | |
| | | | | /A]CGAGTTGGTGG | | |
| | | | | TTGCTCCACAAGA | | |
| | | | | C | | |
| 59 | Chr05 | 41853287 | 0 cM | AGTGTTGAAGCTT | G | A |
| | | | | GCTCGAGTTGGT[G | | |
| | | | | /A]GTTGCTCCACA | | |
| | | | | AGACCTAAAGGTC | | |
| | | | | C | | |
| 60 | Chr05 | 41853304 | 0 cM | GAGTTGGTGGTTG | T | C |
| | | | | CTCCACAAGACC[T | | |
| | | | | /C]AAAGGTCCTTT | | |
| | | | | CTGTTGAAACAAC | | |
| | | | | A | | |
| 61 | Chr05 | 41853319 | 0 cM | CCACAAGACCTAA | T | C |
| | | | | AGGTCCTTTCTG[T/ | | |
| | | | | C]TGAAACAACAC | | |
| | | | | AAAGATATAGCGA | | |
| | | | | T | | |
| 62 | Chr05 | 41853357 | 0 cM | AAGATATAGCGAT | G | T |
| | | | | GTATTCCAATAG[G | | |
| | | | | /T]TGGTCTTGGTCC | | |
| | | | | CACATATGAAACA | | |
| 63 | Chr05 | 41853361 | 0 cM | TATAGCGATGTAT | T | C |
| | | | | TCCAATAGGTGG[T | | |
| | | | | /C]CTTGGTCCCAC | | |
| | | | | ATATGAAACAATC | | |
| | | | | T | | |
| 64 | Chr05 | 41853377 | 0 cM | AATAGGTGGTCTT | G | A |
| | | | | GGTCCCACATAT[G | | |
| | | | | /A]AAACAATCTCT | | |
| | | | | TTTCCTGCAAAGA | | |
| | | | | A | | |
| 65 | Chr05 | 41853464 | 0 cM | TCCCTGCAAAGAA | G | T |
| | | | | GACCATTTACTT[G | | |
| | | | | /T]ATTAATTATTAG | | |
| | | | | ATTTCTTTGTTCC | | |
| 66 | Chr05 | 41853495 | 0 cM | TTATTAGATTTCTT | A | T |
| | | | | TGTTCCGCATG[A/ | | |
| | | | | T]ATCAAATTATTA | | |
| | | | | TTGTTTACAAGAA | | |
| 67 | Chr05 | 41853526 | 0 cM | ATTATTATTGTTTA | T | C |
| | | | | CAAGAAATCAA[T/ | | |
| | | | | C]GTTAACTGTTGA | | |
| | | | | AAGTAGGAAAAAT | | |
| 68 | Chr05 | 41853533 | 0 cM | TTGTTTACAAGAA | T | A |
| | | | | ATCAATGTTAAC[T | | |
| | | | | /A]GTTGAAAGTAG | | |
| | | | | GAAAAATTAATCT | | |
| | | | | A | | |
| 69 | Chr05 | 41853534 | 0 cM | TGTTTACAAGAAA | G | A |
| | | | | TCAATGTTAACT[G | | |
| | | | | /A]TTGAAAGTAGG | | |
| | | | | AAAAATTAATCTA | | |
| | | | | A | | |
| 70 | Chr05 | 41853563 | 0 cM | AAAGTAGGAAAA | C | A |
| | | | | ATTAATCTAATAT[ | | |
| | | | | C/A]CTGTATGCTA | | |
| | | | | GATGCGAAGATTA | | |
| | | | | TA | | |
| 71 | Chr05 | 41853566 | 0 cM | GTAGGAAAAATTA | G | A |
| | | | | ATCTAATATCCT[G | | |
| | | | | /A]TATGCTAGATG | | |
| | | | | CGAAGATTATATT | | |
| | | | | T | | |
| 72 | Chr05 | 41853578 | 0 cM | AATCTAATATCCT | C | T |
| | | | | GTATGCTAGATG[C | | |
| | | | | /T]GAAGATTATAT | | |
| | | | | TTTAGAAATGACA | | |
| | | | | G | | |
| 73 | Chr05 | 41853596 | 0 cM | CTAGATGCGAAGA | A | C |
| | | | | TTATATTTTAGA[A/ | | |
| | | | | C]ATGACAGGACC | | |
| | | | | AAAGAAAGGTGAT | | |
| | | | | A | | |
| 74 | Chr05 | 41853601 | 0 cM | TGCGAAGATTATA | C | A |
| | | | | TTTTAGAAATGA[C | | |
| | | | | /A]AGGACCAAAGA | | |
| | | | | AAGGTGATAATTC | | |
| | | | | A | | |
| 75 | Chr05 | 41853647 | 0 cM | ATTCAAAATATTT | T | C |
| | | | | TTACTTTGGAGT[T/ | | |
| | | | | C]GGAGAAATATT | | |
| | | | | GCTTCATGTTTGA | | |
| | | | | C | | |
| 76 | Chr05 | 41853660 | 0 cM | TTACTTTGGAGTT | C | A |
| | | | | GGAGAAATATTG[ | | |
| | | | | C/A]TTCATGTTTGA | | |
| | | | | CACATTTGCTTGT | | |
| | | | | G | | |
| 77 | Chr05 | 41853677 | 0 cM | AAATATTGCTTCA | T | G |
| | | | | TGTTTGACACAT[T/ | | |
| | | | | G]TGCTTGTGATGG | | |
| | | | | AATTCTGAAATTT | | |
| 78 | Chr05 | 41853747 | 0 cM | GGCACGTGTGATG | G | T |
| | | | | ATCTCATAAAAG[ | | |
| | | | | G/T]AGATTCCCTA | | |
| | | | | GCTAACACATGTT | | |
| | | | | AT | | |
| 79 | Chr05 | 41853791 | 0 cM | ATGTTATCTAAAT | G | T |
| | | | | TGGTGCATGCAC[G | | |
| | | | | /T]GAGAAAATCTA | | |
| | | | | AAATTAATTTATT | | |
| | | | | G | | |
| 80 | Chr05 | 41854089 | 0 cM | CCTGAAATTCTGT | C | T |
| | | | | GATTTTTTTTTC[C/ | | |
| | | | | T]AGAAGAAATCT | | |
| | | | | GAAACATGCATGC | | |
| | | | | G | | |
| 81 | Chr05 | 41854214 | 0 cM | TGTTTCTGAAATC | G | A |
| | | | | GAGAGTCAGTGC[ | | |
| | | | | G/A]ATGGACGCCC | | |
| | | | | AAAAAATTAAAAA | | |
| | | | | AC | | |
| 82 | Chr05 | 41854227 | 0 cM | GAGAGTCAGTGCG | A | G |
| | | | | ATGGACGCCCAA[ | | |
| | | | | A/G]AAATTAAAAA | | |
| | | | | ACATGATGTTTTA | | |
| | | | | GC | | |
| 83 | Chr05 | 41854377 | 0 cM | TATATGAAGTAGA | T | G |
| | | | | CCAAAAGATTTC[T | | |
| | | | | /G]TTTACTACCGC | | |
| | | | | GAAATGGGTTTGT | | |
| | | | | T | | |
| 84 | Chr05 | 41854422 | 0 cM | TTTGTTCCTCCAGG | A | C |
| | | | | TATGTCAACTA[A/ | | |
| | | | | C]GATCCTGGAAA | | |
| | | | | ATAAGTGGAAACA | | |
| | | | | G | | |
| 85 | Chr05 | 41854447 | 0 cM | AGATCCTGGAAAA | G | T |
| | | | | TAAGTGGAAACA[ | | |
| | | | | G/T]CAAGATCATA | | |
| | | | | TCAGTTTATATAT | | |
| | | | | AT | | |
| 86 | Chr05 | 41854473 | 0 cM | CAAGATCATATCA | G | A |
| | | | | GTTTATATATAT[G/ | | |
| | | | | A]GAGTTAGAAAC | | |
| | | | | TAAGAATAGGAAG | | |
| | | | | A | | |
| 87 | Chr05 | 41854533 | 0 cM | AATTTACTAACCA | G | A |
| | | | | ATGTATCCACTC[G | | |
| | | | | /A]CGCATGCTTGG | | |
| | | | | TTCACTGGGGCTA | | |
| | | | | G | | |
| 88 | Chr05 | 41854669 | 0 cM | GAGATCTCTCAAC | G | T |
| | | | | AGAAACATGAAT[ | | |
| | | | | G/T]AATACCAGTG | | |
| | | | | TGTAGAGGTTATC | | |
| | | | | CA | | |
| 89 | Chr05 | 41854717 | 0 cM | CCATGTTGCCGGT | A | G |
| | | | | GAGGGTGAGCTT[ | | |
| | | | | A/G]GGAGGGCTGA | | |
| | | | | TGGCAATGGATGG | | |
| | | | | CT | | |
| 90 | Chr05 | 41854732 | 0 cM | GGGTGAGCTTAGG | A | G |
| | | | | AGGGCTGATGGC[ | | |
| | | | | A/G]ATGGATGGCT | | |
| | | | | TGATGTCACAGCC | | |
| | | | | AT | | |
| 91 | Chr05 | 41854786 | 0 cM | TGACGTGCTTCGA | G | A |
| | | | | CCCAAAGTAAAC[ | | |
| | | | | G/A]GACATGTTGA | | |
| | | | | CTGAAGGAATGAA | | |
| | | | | CA | | |
| 92 | Chr05 | 41854798 | 0 cM | ACCCAAAGTAAAC | T | A |
| | | | | GGACATGTTGAC[T | | |
| | | | | /A]GAAGGAATGAA | | |
| | | | | CATGTCTACCACA | | |
| | | | | T | | |
| 93 | Chr05 | 42136562 | 1 cM | ATTACACCGTGGA | C | T |
| | | | | CGATATTATAAT[C | | |
| | | | | /T]CAATAATATAC | | |
| | | | | TACTGTAGCTACAA | | |

Physical Positions based on BLAST to Glycine max Wm82.a2.v1; available at soybase.org or phytozome-next.jgi.doe.gov

### Example 6

This example demonstrates the isolation of an MFT mutant by searching a sequenced mutant library for mutations in the MFT gene.

Ethyl methanesulphonate (EMS) is a chemical mutagen which is used frequently to develop high density mutant populations. An EMS-induced mutant population was developed by treating soybean variety seeds from an elite soybean variety with EMS. Single seed was harvested from individual M1 plants and propagated to generate M2 lines. About 1200 M2 lines were whole genome sequenced to find mutations in soybean genome. On average, about 4000 mutations per M2 line altering an amino acid residue in a coding region were identified by comparing the mutant sequence to the wild-type elite soybean variety reference genome. By searching for MFT genes in the sequenced mutant library, MFT mutants are identified. Once a mutant is identified, seed composition can be determined by NIR. If the mutant shows a high oil trait, MFT gene-specific molecular markers or MFT flanking molecular markers can be developed and used in backcrossing and breeding. In addition to our internal sequenced mutant library, a public sequenced soybean mutant library is also available (Zhang, M., Zhang, X., Jiang, X., Qiu, L., Jia, G., Wang, L., Ye, W. and Song, Q. (2022) iSoybean: A database for the mutational fingerprints of soybean. Plant Biotechnol J., doi.org/10.1111/pbi.13844). By searching the public sequenced mutant library database (isoybean.org), new MFT mutant alleles can be identified. The identified MFT mutant alleles can be integrated into an elite soybean variety to increase seed oil content by marker assisted backcrossing.

All publications and patent applications in this specification are indicative of the level of ordinary skill in the art to which this invention pertains. All publications and patent applications are herein incorporated by reference to the same extent as if each individual publication or patent application was specifically and individually indicated by reference.

Unless defined otherwise, all technical and scientific terms used herein have the same meaning as commonly understood by one of ordinary skill in the art to which this invention belongs. Unless mentioned otherwise, the techniques employed or contemplated herein are standard methodologies well known to one of ordinary skill in the art. The materials, methods and examples are illustrative only and not limiting.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

Units, prefixes and symbols may be denoted in their SI accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

1. A soybean cell having an increased oil content and comprising a modified MFT gene coding sequence encoding a modified MFT polypeptide comprising an amino acid sequence that is at least 95% identical to SEQ ID NO: 2 and comprises a non-threonine at the amino acid residue corresponding to position 82 of SEQ ID NO: 2.

2. The soybean cell of claim 1, wherein the modified MFT polypeptide sequence comprises a glycine, asparagine, glutamine, alanine, serine, or cysteine at the amino acid residue corresponding to position 82 of SEQ ID NO: 2.

3. The soybean cell of claim 1, wherein the modified MFT polypeptide further comprises a non-leucine at the amino acid residue corresponding to position L140 of SEQ ID NO: 2.

4. The soybean cell of claim 4, wherein the modified MFT polypeptide sequence comprises a glycine, asparagine, glutamine, alanine, serine, cysteine, or threonine at the amino acid residue corresponding to position L140 of SEQ ID NO: 2.

5. (canceled)

6. A soybean plant comprising the soybean cell of claim 1.

7. A soybean seed comprising the soybean cell of claim 1.

8. The soybean seed of claim 7, wherein the oil content of the soybean seed is increased by at least a 1 percentage point, the protein content of the soybean seed is increased by at least a 0.25 percentage point, or a combination thereof, as compared to a control soybean seed when measured at 13% moisture content.

9-18. (canceled)

19. A method for producing a soybean plant having high seed oil, the method comprising:
a. genotyping a soybean population comprising a plurality of soybean plants or soybean germplasm for the presence of at least one marker genetically linked to a locus comprising or corresponding to an MFT gene having at least 95% identity to SEQ ID NO: 7, the at least one marker detecting a modification in the MFT gene;
b. selecting from the soybean population one or more soybean plants or soybean germplasm comprising the at least one marker; and
c. crossing the selected soybean plant or soybean germplasm with a second soybean plant or soybean germplasm to produce a progeny population, wherein at least one soybean plant or soybean germplasm of the progeny population comprises the at least one marker and has high seed oil as compared to a seed of a control plant.

20. The method of claim 19, wherein the at least one marker comprises an insertion, deletion, polymorphism, or any combination thereof in a coding sequence or regulatory sequence of the MFT gene.

21-22. (canceled)

23. The method of claim 19, wherein the at least one marker comprises a polymorphism corresponding to a CC at marker S101AY8-00-Q002, a T at marker S2000A7-001-Q001, a 4 base pair deletion at marker S2000A3-001-Q001, a 1 base pair deletion at marker S2000A4-001-Q001, and a G insertion at marker S2000A5-001-Q001.

24. The method of claim 19, further comprising detecting a second marker genetically linked to the locus comprising or corresponding to the MFT gene.

25. The method of claim 19, wherein genotyping comprises amplifying a nucleic acid sequence comprising the at least one marker and detecting the resulting amplified nucleic acid comprising the marker.

26. The method of claim 25, wherein the amplified nucleic acid comprises at least a portion of a sequence corresponding to SEQ ID NO: 8, 18, 28, 34, or 40.

27. The method of claim 25, wherein the amplifying the nucleic acid sequence comprises providing nucleic acid primers, wherein the nucleic acid primers comprise one or more of SEQ ID NOs: 10, 11, 20, 21, 30, 31, 36, 37, 42, and 43.

28. The method of claim 25, wherein detecting the resulting amplified nucleic acid further comprises hybridizing the resulting amplified nucleic acid with one or more nucleic acid probes, wherein the nucleic acid probes comprise one or more of SEQ ID NOs: 9, 19, 29, 35 and 41.

29-38. (canceled)

39. A method of introgressing a high soybean seed oil MFT allele into a soybean plant, the method comprising:
a. crossing a first soybean plant with a second soybean plant to produce a progeny population, the first soybean plant comprising a high soybean seed oil MFT allele comprising a modification of an MFT gene comprising a polynucleotide sequence having at least 95% sequence identity with SEQ ID NO: 7, the modification decreasing the expression or activity of an MFT polypeptide encoded by the MFT gene;
b. genotyping the progeny population for the presence of at least one marker genetically linked to the high soybean seed oil MFT allele; and
c. selecting progeny that comprise the at least one marker to obtain soybean plants comprising the high soybean seed oil MFT allele.

40. The method of claim 39, wherein the modification is a polymorphism that decreases expression of a polypeptide encoded by the MFT gene compared to a wild-type polypeptide; or decreases activity of a polypeptide encoded by the MFT gene, compared to a wild-type polypeptide.

41. (canceled)

42. The method of claim 39, wherein a seed of a soybean plant selected from the progeny population has an oil content that is increased by at least a 1 percentage point, a protein content that is increased by at least a 0.25 percentage point, or a combination thereof, as compared to a control soybean seed when measured at 13% moisture content.

43. The method of claim 39, wherein the at least one marker genetically linked to the high oil MFT allele is within 20 centimorgans of the high oil MFT allele.

44. The method of claim 39, wherein the at least one marker comprises a polymorphism corresponding to a CC at marker S101AY8-00-Q002, a T at marker S2000A7-001-Q001, a 4 base pair deletion at marker S2000A3-001-Q001, a 1 base pair deletion at marker S2000A4-001-Q001, a G insertion at marker S2000A5-001-Q001, a T at position 38012490 on Chr05, an A at position 39924818 on Chr05, a T at position 40892689 on Chr05, a C at position 41265253 on Chr05, a G at position 41673315 on Chr05, and a C at position 42136562 on Chr05.
